SEQUENCE LISTING (13 sequences)

SEQ ID NO: 1 - protein, 195 aa, Homo sapiens
MAAEDVVATGADPSDLESGGLLHEIFTSPLNLLLLGLCIFLLYKIVRGDQPAASGDSDDDEPPPLPRLKRRDFTPAELRRFDGVQDPRILMAINGKVFDVTKGRKFYGPEGPYGVFAGRDASRGLATFCLDKEALKDEYDDLSDLTAAQQETLSDWESQFTFKYHHVGKLLKEGEEPTVYSDEEEPKDESARKND

SEQ ID NO: 2 - protein, 1075 aa, Homo sapiens
MKGTCVIAWLFSSLGLWRLAHPEAQGTTQCQRTEHPVISYKEIGPWLREFRAKNAADFSQLTFDPGQKELVVGARNYLFRLQLEDLSLIQAVEWECDEATKKACYSKGKSKEECQNYIRVLLVGGDRLFTCGTNAFTPVCTNRSLSNLAEIHDQISGMARCPYSPQHNSTALLTAGGELYAATAMDFPGRDPAIYRSLGILPPLRTAQYNSKWLNEPNFVSSYDIGNFTYFFFRENAVEHDCGKTVFSRAARVCKNDIGGRFLLEDTWTTFMKARLNCSRPGEVPFYYNELQSTFFLPELDLIYGIFTTNVNSIAASAVCVFNLSAIAQAFSGPFKYQENSRSAWLPYPNPNPHFQCGTVDQGLYVNLTERNLQDAQKFILVHEVVQPVTTVPSFMEDNSRFSHVAVDVVQGREALVHIIYLATDYGTIKKVRVPLNQTSSSCLLEEIELFPERRREPIRSLQILHSQSVLFVGLREHVVKIPLKRCQFYRTRSTCIGAQDPYCGWDVVMKKCTSLEESLSMTQWEQSISACPTRNLTVDGHFGVWSPWTPCTHTDGSAVGSCLCRTRSCDSPAPQCGGWQCEGPGMEIANCSRNGGWTPWTSWSPCSTTCGIGFQVRQRSCSNPTPRHGGRVCVGQNREERYCNEHLLCPPHMFWTGWGPWERCTAQCGGGIQARRRICENGPDCAGCNVEYQSCNTNPCPELKKTTPWTPWTPVNISDNGDHYEQRFRYTCKARLADPNLLEVGRQRIEMRYCSSDGTSGCSTDGLSGDFLRAGRYSAHTVNGAWSAWTSWSQCSRDCSRGIRNRKRVCNNPEPKYGGMPCLGPSLEYQECNTLPCPVDGVWSCWSPWTKCSATCGGGHYMRTRSCSNPAPAYGGDICLGLHTEEALCNTQPCPESWSEWSDWSECEASGVQVRARQCILLFPMGSQCSGNTTESRPCVFDSNFIPEVSVARSSSVEEKRCGEFNMFHMIAVGLSSSILGCLLTLLVYTYCQRYQQQSHDATVIHPVSPAPLNTSITNIHINKLDKYDSVEAIKAFNKNNLILEERNKYFNPHLTGKTYSNAYFTDLNNYDEY

SEQ ID NO: 3 - protein, 524 aa, Homo sapiens
MTEDKVTGTLVFTVITAVLGSFQFGYDIGVINAPQQVIISHYRHVLGVPLDDRKAINNYVINSTDELPTISYSMNPKPTPWAEEETVAAAQLITMLWSLSVSSFAVGGMTASFFGGWLGDTLGRIKAMLVANILSLVGALLMGFSKLGPSHILIIAGRSISGLYCGLISGLVPMYIGEIAPTALRGALGTFHQLAIVTGILISQIIGLEFILGNYDLWHILLGLSGVRAILQSLLLFFCPESPRYLYIKLDEEVKAKQSLKRLRGYDDVTKDINEMRKEREEASSEQKVSIIQLFTNSSYRQPILVALMLHVAQQFSGINGIFYYSTSIFQTAGISKPVYATIGVGAVNMVFTAVSVFLVEKAGRRSLFLIGMSGMFVCAIFMSVGLVLLNKFSWMSYVSMIAIFLFVSFFEIGPGPIPWFMVAEFFSQGPRPAALAIAAFSNWTCNFIVALCFQYIADFCGPYVFFLFAGVLLAFTLFTFFKVPETKGKSFEEIAAEFQKKSGSAHRPKAAVEMKFLGATETV

SEQ ID NO: 4 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEVTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 5 - protein, 1542 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCWIFVFQLTPVLVSVVTFSVYVLVDSNNILDAQKAFTSITLFNILRFPLSMLPMMISSMLQASVSTERLEKYLGGDDLDTSAIRHDCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEAWHDGSEEEDDDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRSWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGWGRTGAGKSSLTNCLFRILEAAGGQIIIDGVDIASIGLHDLREKLTIIPQDPILFSGSLRMNLDPFNNYSDEEIWKALELAHLKSFVASLQLGLSHEGTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 6 - protein, 1545 aa, Homo sapiens [sequence omitted]

SEQ ID NO: 7 - protein, 1545 aa, Homo sapiens
MLEKFCNSTFWNSSFLDSPEADLPLCFEQTVLVWIPLGFLWLLAPWQLLHVYKSRTKRSSTTKLYLAKQVFVGFLLILAAIELALVLTEDSGQATVPAVRYTNPSLYLGTWLLVLLIQYSRQWCVQKNSWFLSLFWILSILCGTFQFQTLIRTLLQGDNSNLAYSCLFFISYGFQILILIFSAFSENNESSNNPSSIASFLSSITYSWYDSIILKGYKRPLTLEDVWEVDEEMKTKTLVSKFETHMKRELQKARRALQRRQEKSSQQNSGARLPGLNKNQSQSQDALVLEDVEKKKKKSGTKKDVPKSWLMKALFKTFYMVLLKSFLLKLVNDIFTFVSPQLLKLLISFASDRDTYLWIGYLCAILLFTAALIQSFCLQCYFQLCFKLGVKVRTAIMASVYKKALTLSNLARKEYTVGETVNLMSVDAQKLMDVTNFMHMLWSSVLQIVLSIFFLWRELGPSVLAGVGVMVLVIPINAILSTKSKTIQVKNMKNKDKRLKIMNEILSGIKILKYFAWEPSFRDQVQNLRKKELKNLLAFSQLQCVVIFVFQLTPVLVSVVTFSVYVLVDSNNILDAQKAFTSITLFNILRFPLSMLPMMISSMLQASVSTERLEKYLGGDDLDTSAIRHSCNFDKAMQFSEASFTWEHDSEATVRDVNLDIMAGQLVAVIGPVGSGKSSLISAMLGEMENVHGHITIKGTTAYVPQQSWIQNGTIKDNILFGTEFNEKRYQQVLEACALLPDLEMLPGGDLAEIGEKGINLSGGQKQRISLARATYQNLDIYLLDDPLSAVDAHVGKHIFNKVLGPNGLLKGKTRLLVTHSMHFLPQVDEIVVLGNGTIVEKGSYSALLAKKGEFAKNLKTFLRHTGPEEEATVHDGSEEEADDYGLISSVEEIPEDAASITMRRENSFRRTLSRSSRSNGRHLKSLRNSLKTRNVNSLKEDEELVKGQKLIKKEFIETGKVKFSIYLEYLQAIGLFSIFFIILAFVMNSVAFIGSNLWLSAWTSDSKIFNSTDYPASQRDMRVGVYGALGLAQGIFVFIAHFWSAFGFVHASNILHKQLLNNILRAPMRFFDTTPTGRIVNRFAGDISTVDDTLPQSLRTWITCFLGIISTLVMICMATPVFTIIVIPLGIIYVSVQMFYVSTSRQLRRLDSVTRSPIYSHFSETVSGLPVIRAFEHQQRFLKHNEVRIDTNQKCVFSWITSNRWLAIRLELVGNLTVFFSALMMVIYRDTLSGDTVGFVLSNALNITQTLNWLVRMTSEIETNIVAVERITEYTKVENEAPWVTDKRPPPDWPSKGKIQFNNYQVRYRPELDLVLRGITCDIGSMEKIGVVGRTGAGKSSLTNCLFRILEAAGGQIIIDGVDIASIGLHDLREKLTIIPQDPILFSGSLRMNLDPFNNYSDEEIWKALELAHLKSFVASLQLGLSHEGTEAGGNLSIGQRQLLCLGRALLRKSKILVLDEATAAVDLETDNLIQTTIQNEFAHCTVITIAHRLHTIMDSDKVMVLDNGKIIECGSPEELLQIPGPFYFMAKEAGIENVNSTKF

SEQ ID NO: 8 - protein, 657 aa, Homo sapiens [sequence omitted]

SEQ ID NO: 9 - DNA, 1890 bp, Homo sapiens
gacccacgcgtccggggaggagaaagtggcgagttccggatccctgcctagcgcggcccaacctttactccagagatcatggctgccgaggatgtggtggcgactggcgccgacccaagcgatctggagagcggcgggctgctgcatgagattttcacgtcgccgctcaacctgctgctgcttggcctctgcatcttcctgctctacaagatcgtgcgcggggaccagccggcggccagcggcgacagcgacgacgacgagccgccccctctgccccgcctcaagcggcgcgacttcacccccgccgagctgcggcgcttcgacggcgtccaggacccgcgcatactcatggccatcaacggcaaggtgttcgatgtgaccaaaggccgcaaattctacgggcccgaggggccgtatggggtctttgctggaagagatgcatccaggggccttgccacattttgcctggataaggaagcactgaaggatgagtacgatgacctttctgacctcactgctgcccagcaggagactctgagtgactgggagtctcagttcactttcaagtatcatcacgtgggcaaactgctgaaggagggggaggagcccactgtgtactcagatgaggaagaaccaaaagatgagagtgcccggaaaaatgattaaagcattcagtggaagtatatctatttttgtattttgcaaaatcatttgtaacagtccactctgtctttaaaacatagtgattacaatatttagaaagttttgagcacttgctataagttttttaattaacatcactagtgacactaataaaattaacttcttagaatgcatgatgtgtttgtgtgtcacaaatccagaaagtgaactgcagtgctgtaatacacatgttaatactgtttttcttctatctgtagttagtacaggatgaatttaaatgtgtttttcctgagagacaaggaagacttgggtatttcccaaaacaggtaaaaatcttaaatgtgcaccaagagcaaaggatcaacttttagtcatgatgttctgtaaagacaacaaatccctttttttttctcaattgacttaactgcatgatttctgttttatctacctctaaagcaaatctgcagtgttccaaagactttggtatggattaagcgctgtccagtaacaaaatgaaatctcaaaacagagctcagctgcaaaaaagcatattttctgtgtttctggactgcactgttgtccttgccctcacatagacactcagacaccctcacaaacacagtagtctatagttaggattaaaataggatctgaacattcaaaagaaagctttggaaaaaaagagctggctggcctaaaaacctaaatatatgatgaagattgtaggactgtcttcccaagccccatgttcatggtggggcaatggttatttggttattttactcaattggttactctcatttgaaatgagggagggacatacagaataggaacaggtgtttgctctcctaagagccttcatgcacacccctgaaccacgaggaaacagtacagtcgctagtcaagtggtttttaaagtaaagtatattcataaggtaacagttattctgttgttataaaactatacccactgcaaaagtagtagtcaagtgtctaggtctttgatattgctcttttggttaacactaagcttaagtagactatacagttgtatgaatttgtaaaagtatatgaacacctagtgagatttcaaacttgtaattgtggttaaatagtcattgtattttcttgtgaactgtgttttatgattttacctcaaatcagaaaacaaaatgatgtgctttggtcagttaataaaaatggttttacccactaaaaaaaaaaaaa

SEQ ID NO: 10 - DNA, 8056 bp, Homo sapiens
ctgctctccctgagcccgctcccgagcgctgctttcccgccgcgggtgggcttcgcagcctcaggccagccgcggcccttggcccgctgcagccccggccctccaccttccccgtgcaggggcggcccggccagtgtcgctcatcccgggacgctcccttctcccacccaggactgccccgcggagctggcttggacacccaactttgccacctcgagggtcgtctctgctgggcgcgaacctgcccacccaccggttggccgcgcgctcggggaccgtgctcgtggcccccaagccggtgcccccattctggaactcagcgagtagggggcggctctggggaagtggcagggggcggctgcagctgctgcctccacttccctagccaggtgctgaagaggatcctcggagccgctctggcccccaggcgctggatgactggcaccagcgctcctcgcacctgtgttggtgtgtgagacttgggctggagtgcccacgtggctgtggagtcagtgtgattcatgattgaggaaacgcgtcctccatcctctctctccttggcactttccacacatgaggagaagaagagcttctgtttagaagacacgtgcccagagtcagaggccccttgcccaccatgaagggaacctgtgttatagcatggctgttctcaagcctggggctgtggagactcgcccacccagaggcccagggtacgactcagtgccagagaaccgagcatccagtcatctcctataaagaaattggcccctggttacgggagttcagagcgaagaatgctgcggatttctcgcagttaacatttgacccaggacagaaagaacttgttgtaggagcaagaaactacctcttcaggttacagcttgaggatctgtctcttatccaggctgtggaatgggagtgtgatgaagctaccaaaaaggcctgttacagcaaaggcaaatcaaaggaggaatgtcagaactacatccgggtgcttctggtgggtggcgaccggttattcacctgtgggaccaatgcattcacgcctgtctgcaccaaccgctcgttgagcaacctggctgagatccatgatcagatcagtggcatggcccgctgtccctacagtccccagcacaattccacagcgctcctcacagctggtggggagctctatgctgctacagccatggattttccaggacgtgatcctgccatttaccgaagcctaggcattttacctcctctccgcacggcgcagtacaactccaaatggctcaatgagccaaactttgtgtcatcttatgacatcggaaattttacctacttctttttccgagaaaatgcagtagagcatgactgtgggaaaacagtgttctccagagctgcccgggtgtgcaagaacgatattggtgggcgcttcctgctggaagacacctggaccacattcatgaaggctcgcctgaactgctcccgtcctggggaagtccccttttactacaacgaattgcagagtactttcttcctgcctgagctggatttgatctatggcatctttaccaccaatgtgaacagcattgcggcctcagctgtgtgcgtcttcaacctgagcgccatcgcgcaggccttctctgggcccttcaagtaccaagaaaactcgcgctcggcctggctaccgtatcccaacccaaacccccacttccagtgtggcaccgtggaccagggcctgtacgtgaacctgaccgagagaaatctgcaggatgctcagaagttcattctggtgcatgaggtggtacagccagtgaccacagtgccctccttcatggaggacaatagccgcttttcccacgtggcagtcgacgtggtgcagggcagagaagcgctcgtccacatcatctatttggccacagattacggaaccattaagaaagtgcgggtacccctgaatcagacctcaagcagctgtttgctggaagagattgagctcttccctgagaggcggagggagcccatcaggagcctgcagatcctgcacagccagagtgtcctgttcgtggggctgcgggagcacgtggtcaagatccccctgaagaggtgccagttctaccgcacacgcagcacctgcattggggcccaggacccttactgtggctgggatgtggtaatgaagaaatgcacaagcctggaggagagcctgagcatgacgcagtgggaacagagcatctctgcgtgtccgaccaggaatctcaccgtggatgggcactttggtgtgtggtctccgtggacgccttgcacgcacacagatggcagcgccgtgggatcctgcctctgtcgaacccgctcctgcgacagcccggccccgcagtgtggtggctggcagtgcgagggccctggcatggagatcgccaactgttccaggaacggaggctggactccctggacctcgtggtctccctgcagcactacctgtgggatcggcttccaggtgcggcagcgctcctgcagcaaccccactcccaggcacgggggccgggtgtgcgtgggacagaaccgcgaggaaagatactgcaatgaacatttgctatgtcccccacacatgttctggacaggctggggtccttgggaacggtgcacagcccaatgcgggggtggcattcaagctcgccgcaggatctgtgagaatgggcctgactgtgcaggctgcaatgtggagtaccagtcttgcaacaccaacccgtgtcctgagctgaagaagaccacgccctggacaccctggacacctgtcaacatctctgacaacggcgaccactatgagcaacgattccgatacacatgcaaagcccgcctggctgatccgaatttgctggaagtgggaagacagagaatcgaaatgcggtactgttctagcgacggcaccagtggctgctccacagatgggctttctggggatttcctgcgtgctgggagatactctgcccacacggtcaacggggcttggtcagcctggacgtcgtggtcacagtgcagccgtgactgcagcaggggcattcggaaccggaagcgtgtttgcaacaaccccgaacccaagtatgggggaatgccttgccttggcccatctctggaataccaggaatgcaacactttgccctgcccagtggatggcgtgtggtcttgctggtccccctggacaaaatgttcagcaacatgcggcggtggacactatatgaggacccgctcttgctccaatccagccccggcctatggaggggacatctgcctggggctgcacacagaagaggcactctgcaacacgcagccctgcccagagagctggtcggagtggtcggactggtctgagtgtgaagcctctggcgtccaagtccgcgcccgccagtgcatcctcctgttccccatgggcagccagtgctccgggaacaccacggagagccggccgtgtgtgtttgactctaatttcatcccagaagtatctgtggcaagatccagtagcgtagaagagaaaaggtgtggagagttcaacatgttccacatgatcgccgtggggctgagcagctccatcctcggctgcctcctcaccctgctcgtctatacttactgccagcggtaccagcagcaatcccacgatgcgactgtcatccaccccgtctcacctgccccccttaataccagcataaccaaccacatcaacaaactggacaagtacgactcggtggaggccatcaaggcatttaacaaaaacaacttgatcctagaggaaagaaacaaatacttcaacccacatctcactgggaagacctattctaatgcctactttacagatctcaataattatgatgagtactaacagctttcatgttttgggcttcttgtaaatccccagttcctcaaggcctgtgccccatgactgcccatgtttctgaggcttcagagtcgaagtttggatacatttcaagtgcatttcaagccaccagagtgtcccattggtgccaaaaatacacgtctttaaaagcaacaaaaattgaaataagacatcgtgaaaatcttgaccattgttgattgagccagggtggtgaagtttttaattggtgttcatcctatttttctgacaagtccattggtttgttttttgagcattattttataaatgtgccacccacattggaaggagtctttctttagaactttggagtgtaaatcttcatgatgttgtaaattcaagaaaataggcactttctctgaaagacctgctccttccacaagaagtgcataggtccataatatttcataaaatgaagaaaaagaatgtggccaaacaattattcaccatggattgcccaactttccaaatctggataaagctgtgggattcttggaagcagcttgagtgttttcatcttgcctgggaagcccaggaattccacctggtccacaccggcagaagttacagtagactgtgaggcaccccaccttgctcctgatgcagtttctgtgccattgctggtgctggtggaggcagcggagcagaggctcaggcacaatgaagcgtggatgtgttctgcaggttgctgcaaaactcaccttattctgacttttggatttcatggcattccaggaagctccttgccatgctgttggcctggaagtccacctgtctggtccatagtgacgtcctgaagagccagtctgtaaaataaccaaccacttacttagcgtttggatagactccatgccttctctctccctgcaaagaaaaatcttgaacatttatgatgtcaattagtgaaagtattgaaaatactaaattataactaaaagcaactttttatgttattgaaaatattgaaagaactgatattaatgataatcattttatttttcatctctctgatatacccaatgtgtggcaatcagcccctaccatgagcattaatgccatgtaaagctggctttctggagtctgccaggtccacacgaagtgctacccccagtttctgctgttacttgctgcctccaggccaggggagcaggagatgctcagctctggtgccttttcttttatttcagtgctgccttcccacccacccagtccattcaccctcacctctccctgcatggaggcaacagcatttccaagatgtaccttgggaagtcaggatagctggaagtaagggtttttggggatccctgtggtctcttcactgatcatcgataagcatttaagagtgtgctaacaccattcacagaggtccctggaaaaaaatatataattttctcacaaacaaccaatacataacctatggcagttggttgaataattattgaaaaaaagaacacgactgaaaaagttcatctgatgcctctgagcatgtgataaagtcctgggtagacatggaaacgtgttcttacaaatgaaccttcggatctatgaaagaaaatatagtagggggactaaggaacatagaattttattagcatatgtgattttacccttatctttgtttctaatttaaagaaacaatttcagaaagtgttagaagaattctatgtttaataatgaatattgttgagttcaaaatattctcatatacccagatttacagagatgacacagtattgaaatggcaggtgggctctgtaagttatttggggattaatgaaatccagggtcttggcaggggggactcaatgtggccctctcactcatccctcatatgtcaagagtccttcctgtcttatatgcacatgctccaaggtccatactcagtagggggaaatctagccggatggttccccaattgttcctcggctggattttcatcatcagaaatttccaactcatctcatcagttttcttgtaagaaaaaaacatttttcactttgtagcacaattttagttatttttgagtcttcttccctaatagtttgtaagcttaaagggacaattttaattttcccctggttaaagagggattctaatatttccaagaatgttttccactggaattgatgggaaatggtttctaaatggcatcgaagctggtctttaatgatcttttgcaatgacttgggaaaaacaccctccctcactacaggatcttcatgttgttaataatataaacaaacctttgaaattagcattgcaaaagaatcccatttttgtttcgtaccacactgttcaaaggaaaattgtttatctctcttcctttctctctctctcttatttgacagaatagcaagtgtttagataatttcagatatattcctaagtattttaccagctgtggaataagttgtgtttgtccatcactgtgtagctaccgttcacatgttctggctctgtactccacgcttatcgcgtgaaccctcacatgttggactttcacagtgtgatctctcacctgtcttggaaactccttcataaagccgctcttccttaggcctgggctgtttggaagtcctggtgaaactttgcggttcacattttaaattcctgaatgaccttttcattctctctttctttatctttgtttttgtccttcattccctccttttgttcttccttcctgcctttcacgtcacctacttatccgactatttcctccatttgcttcttttctatttgccatcacctgtgacccgagtggaacaaaaaccaacacatagttttaagctacacctttctgcacctgatgtaaaatgacattaatccactatttaacttctaaaattatttctgaatacagttgcagataggctggttttcatgaggaagctcgcttggctttagtcttacatttaaattctttgaaagtggctcccagtgctattcagggtcctttcttgaggccagactcatcaccatctactattgattttacagtgcactgacagtttacaggaaggagaagaacagaatttctgcacactacacaacatgtgggcttcctgtagctccagggacatgtagctttggtgaaactggggttttgtaacctctgaatgatatggacttagtgaattctaaggaactcagggggcacgtggtcaggctccaccgcacagaagagccacagtctccagactcatggcgttccctccagaactccccattcccctctgagcatatttctatgctgctgcttcttgtgacttacatgaggcatctcagcttcttcatgtttgtagggatggttccctaagcctgttcaagtaggctctcactagagttaccattcatatttgagaagaaaagaacatttattaaatgtatgtgtggggctgaggttctaaaggaattgaaaagagacgataaacattgaatgagggtgagggcatccctgctggggagaaacctctgtcccctaggaagagtccgttcatgtcatgtggtttgggatgaaccaggggtctgcccccatcgggtcacaggtgatgccaaatagaaaagaagcaaatggaggaaatagtcggataagtaggtgacgtgaaaggcaggacctggtcaccccagcaagtgctatggacagttcccggaaacggttgcccacttcacaggtccatgggtctgacccttggactctgccaggatcaactgcccagagtgccagagttttagccaaaggtgtacttacttccttatttatctccaaaaggatggaaactgtgggagtcaaagcctattttgctgagtgttcccactggatcctctgtagaattagcaggtcatgctgtcaaaatcatggacaaaggctgggtgcagtggctcatgcctataatcccagcactttgggaggccaaggtgggcggatcacctgagctcaggagtttaagaccagcctgggcaacatggggaaactccatctctacaaaatatacaaaatattagccagccatcgtggtgcgtgcctgtggtcccagtttcttgggaggctgaggcgggagtatcatttgagccaggaggttgaggttgcagtgagctgagatcacatcactgcactccatcctgggtgacagagagagaccctgtctcaaaaaaaaa

SEQ ID NO: 11 - DNA, 3439 bp, Homo sapiens
tctggtttgtaacttatgcctaagggacctgctcccattttctttcctagtggaacaaaggtattgaagccacaggttgctgaggcaaagcacttattgattagattcccatcaatattcagctgccgctgagaagattagacttggactctcaggtctgggtagcccaactcctccctctccttgctcctcctcctgcaatgcataactaggcctaggcagagctgcgaataaacaggcaggagctagtcaggtgcatgtgccacactcacacaagacctggaattgacaggactcccaactagtacaatgacagaagataaggtcactgggaccctggttttcactgtcatcactgctgtgctgggttccttccagtttggatatgacattggtgtgatcaatgcacctcaacaggtaataatatctcactatagacatgttttgggtgttccactggatgaccgaaaagctatcaacaactatgttatcaacagtacagatgaactgcccacaatctcatactcaatgaacccaaaaccaaccccttgggctgaggaagagactgtggcagctgctcaactaatcaccatgctctggtccctgtctgtatccagctttgcagttggtggaatgactgcatcattctttggtgggtggcttggggacacacttggaagaatcaaagccatgttagtagcaaacattctgtcattagttggagctctcttgatggggttttcaaaattgggaccatctcatatacttataattgctggaagaagcatatcaggactatattgtgggctaatttcaggcctggttcctatgtatatcggtgaaattgctccaaccgctctcaggggagcacttggcacttttcatcagctggccatcgtcacgggcattcttattagtcagattattggtcttgaatttatcttgggcaattatgatctgtggcacatcctgcttggcctgtctggtgtgcgagccatccttcagtctctgctactctttttctgtccagaaagccccagatacctttacatcaagttagatgaggaagtcaaagcaaaacaaagcttgaaaagactcagaggatatgatgatgtcaccaaagatattaatgaaatgagaaaagaaagagaagaagcatcgagtgagcagaaagtctctataattcagctcttcaccaattccagctaccgacagcctattctagtggcactgatgctgcatgtggctcagcaattttccggaatcaatggcattttttactactcaaccagcatttttcagacggctggtatcagcaaacctgtttatgcaaccattggagttggcgctgtaaacatggttttcactgctgtctctgtattccttgtggagaaggcagggcgacgttctctctttctaattggaatgagtgggatgtttgtttgtgccatcttcatgtcagtgggacttgtgctgctgaataagttctcttggatgagttatgtgagcatgatagccatcttcctctttgtcagcttctttgaaattgggccaggcccgatcccctggttcatggtggctgagtttttcagtcaaggaccacgtcctgctgctttagcaatagctgcattcagcaattggacctgcaatttcattgtagctctgtgtttccagtacattgcggacttctgtggaccttatgtgtttttcctctttgctggagtgctcctggcctttaccctgttcacattttttaaagttccagaaaccaaaggaaagtcttttgaggaaattgctgcagaattccaaaagaagagtggctcagcccacaggccaaaagctgctgtagaaatgaaattcctaggagctacagagactgtgtaaaaaaaaaaccctgctttttgacatgaacagaaacaataagggaaccgtctgtttttaaatgatgattccttgagcattttatatccacatctttaagtattgttttatttttatgtgctctcatcagaaatgtcatcaaatattaccaaaaaagtatttttttaagttagagaatatatttttgatggtaagactgtaattaagtaaaccaaaaaggctagtttattttgttacactaaagggcaggtggttctaatatttttagctctgttctttataacaaggttcttctaaaattgaagagatttcaacatatcatttttttaacacataactagaaacctgaggatgcaacaaatatttatatatttgaatatcattaaattggaattttcttacccatatatcttatgttaaaggagatatggctagtggcaataagttccatgttaaaatagacaactcttccatttattgcactcagcttttttcttgagtactagaatttgtattttgcttaaaattttacttttgttctgtattttcatgtggaatggattatagagtatactaaaaaatgtctatagagaaaaactttcatttttggtaggcttatcaaaatctttcagcactcagaaaagaaaaccattttagttcctttatttaatggccaaatggtttttgcaagatttaacactaaaaaggtttcacctgatcatatagcgtgggttatcagttaacattaacatctattataaaaccatgttgattcccttctggtacaatcctttgagttatagtttgctttgctttttaattgaggacagcctggttttcacatacactcaaacaatcatgagtcagacatttggtatattacctcaaattcctaataagtttgatcaaatctaatgtaagaaaatttgaagtaaaggattgatcactttgttaaaaatattttctgaattattatgtctcaaaataagttgaaaaggtagggtttgaggattcctgagtgtgggcttctgaaacttcataaatgttcagcttcagacttttatcaaaatccctatttaattttcctggaaagactgattgttttatggtgtgttcctaacataaaataatcgtctcctttgacatttccttctttgtcttagctgtatacagattctagccaaactattctatggccattactaacacgcattgtacactatctatctgcctttacctacataggcaaattggaaatacacagatgattaaacagactttagcttacagtcaattttacaattatggaaatatagttctgatgggtcccaaaagcttagcagggtgctaacgtatctctaggctgttttctccaccaactggagcactgatcaatccttcttatgtttgctttaatgtgtattgaagaaaagcactttttaaaaagtactctttaagagtgaaataattaaaaaccactgaacatttgctttgttttctaaagttgttcacatatatgtaatttagcagtccaaagaacaagaaattgtttcttttc

SEQ ID NO: 12 - DNA, 4868 bp, Homo sapiens
gcggccgcgtctttgttccagacgcagtccaggaatcatgctggagaagttctgcaactctactttttggaattcctcattcctggacagtccggaggcagacctgccactttgttttgagcaaactgttctggtgtggattcccttgggcttcctatggctcctggccccctggcagcttctccacgtgtataaatccaggaccaagagatcctctaccaccaaactctatcttgctaagcaggtattcgttggttttcttcttattctagcagccatagagctggcccttgtactcacagaagactctggacaagccacagtccctgctgttcgatataccaatccaagcctctacctaggcacatggctcctggttttgctgatccaatacagcagacaatggtgtgtacagaaaaactcctggttcctgtccctattctggattctctcgatactctgtggcactttccaatttcagactctgatccggacactcttacagggtgacaattctaatctagcctactcctgcctgttcttcatctcctacggattccagatcctgatcctgatcttttcagcattttcagaaaataatgagtcatcaaataatccatcatccatagcttcattcctgagtagcattacctacagctggtatgacagcatcattctgaaaggctacaagcgtcctctgacactcgaggatgtctgggaagttgatgaagagatgaaaaccaagacattagtgagcaagtttgaaacgcacatgaagagagagctgcagaaagccaggcgggcactccagagacggcaggagaagagctcccagcagaactctggagccaggctgcctggcttgaacaagaatcagagtcaaagccaagatgcccttgtcctggaagatgttgaaaagaaaaaaaagaagtctgggaccaaaaaagatgttccaaaatcctggttgatgaaggctctgttcaaaactttctacatggtgctcctgaaatcattcctactgaagctagtgaatgacatcttcacgtttgtgagtcctcagctgctgaaattgctgatctcctttgcaagtgaccgtgacacatatttgtggattggatatctctgtgcaatcctcttattcactgcggctctcattcagtctttctgccttcagtgttatttccaactgtgcttcaagctgggtgtaaaagtacggacagctatcatggcttctgtatataagaaggcattgaccctatccaacttggccaggaaggagtacaccgttggagaaacagtgaacctgatgtctgtggatgcccagaagctcatggatgtgaccaacttcatgcacatgctgtggtcaagtgttctacagattgtcttatctatcttcttcctatggagagagttgggaccctcagtcttagcaggtgttggggtgatggtgcttgtaatcccaattaatgcgatactgtccaccaagagtaagaccattcaggtcaaaaatatgaagaataaagacaaacgtttaaagatcatgaatgagattcttagtggaatcaagatcctgaaatattttgcctgggaaccttcattcagagaccaagtacaaaacctccggaagaaagagctcaagaacctgctggcctttagtcaactacagtgtgtagtaatattcgtcttccagttaactccagtcctggtatctgtggtcacattttctgtttatgtcctggtggatagcaacaatattttggatgcacaaaaggccttcacctccattaccctcttcaatatcctgcgctttcccctgagcatgcttcccatgatgatctcctccatgctccaggccagtgtttccacagagcggctagagaagtacttgggaggggatgacttggacacatctgccattcgacatgactgcaattttgacaaagccatgcagttttctgaggcctcctttacctgggaacatgattcggaagccacagtccgagatgtgaacctggacattatggcaggccaacttgtggctgtgataggccctgtcggctctgggaaatcctccttgatatcagccatgctgggagaaatggaaaatgtccacgggcacatcaccatcaagggcaccactgcctatgtcccacagcagtcctggattcagaatggcaccataaaggacaacatcctttttggaacagagtttaatgaaaagaggtaccagcaagtactggaggcctgtgctctcctcccagacttggaaatgctgcctggaggagatttggctgagattggagagaagggtataaatcttagtgggggtcagaagcagcggatcagcctggccagagctacctaccaaaatttagacatctatcttctagatgaccccctgtctgcagtggatgctcatgtaggaaaacatatttttaataaggtcttgggccccaatggcctgttgaaaggcaagactcgactcttggttacacatagcatgcactttcttcctcaagtggatgagattgtagttctggggaatggaacaattgtagagaaaggatcctacagtgctctcctggccaaaaaaggagagtttgctaagaatctgaagacatttctaagacatacaggccctgaagaggaagccacagtccatgatggcagtgaagaagaagacgatgactatgggctgatatccagtgtggaagagatccccgaagatgcagcctccataaccatgagaagagagaacagctttcgtcgaacacttagccgcagttctaggtccaatggcaggcatctgaagtccctgagaaactccttgaaaactcggaatgtgaatagcctgaaggaagacgaagaactagtgaaaggacaaaaactaattaagaaggaattcatagaaactggaaaggtgaagttctccatctacctggagtacctacaagcaataggattgttttcgatattcttcatcatccttgcgtttgtgatgaattctgtggcttttattggatccaacctctggctcagtgcttggaccagtgactctaaaatcttcaatagcaccgactatccagcatctcagagggacatgagagttggagtctacggagctctgggattagcccaaggtatatttgtgttcatagcacatttctggagtgcctttggtttcgtccatgcatcaaatatcttgcacaagcaactgctgaacaatatccttcgagcacctatgagattttttgacacaacacccacaggccggattgtgaacaggtttgccggcgatatttccacagtggatgacaccctgcctcagtccttgcgcagctggattacatgcttcctggggataatcagcacccttgtcatgatctgcatggccactcctgtcttcaccatcatcgtcattcctcttggcattatttatgtatctgttcagatgttttatgtgtctacctcccgccagctgaggcgtctggactctgtcaccaggtccccaatctactctcacttcagcgagaccgtatcaggtttgccagttatccgtgcctttgagcaccagcagcgatttctgaaacacaatgaggtgaggattgacaccaaccagaaatgtgtcttttcctggatcacctccaacaggtggcttgcaattcgcctggagctggttgggaacctgactgtcttcttttcagccttgatgatggttatttatagagataccctaagtggggacactgttggctttgttctgtccaatgcactcaatatcacacaaaccctgaactggctggtgaggatgacatcagaaatagagaccaacattgtggctgttgagcgaataactgagtacacaaaagtggaaaatgaggcaccctgggtgactgataagaggcctccgccagattggcccagcaaaggcaagatccagtttaacaactaccaagtgcggtaccgacctgagctggatctggtcctcagagggatcacttgtgacatcggtagcatggagaagattggtgtggtgggcaggacaggagctggaaagtcatccctcacaaactgcctcttcagaatcttagaggctgccggtggtcagattatcattgatggagtagatattgcttccattgggctccacgacctccgagagaagctgaccatcatcccccaggaccccatcctgttctctggaagcctgaggatgaatctcgaccctttcaacaactactcagatgaggagatttggaaggccttggagctggctcacctcaagtcttttgtggccagcctgcaacttgggttatcccacgaagtgacagaggctggtggcaacctgagcataggccagaggcagctgctgtgcctgggcagggctctgcttcggaaatccaagatcctggtcctggatgaggccactgctgcggtggatctagagacagacaacctcattcagacgaccatccaaaacgagttcgcccactgcacagtgatcaccatcgcccacaggctgcacaccatcatggacagtgacaaggtaatggtcctagacaacgggaagattatagagtgcggcagccctgaagaactgctacaaatccctggacccttttactttatggctaaggaagctggcattgagaatgtgaacagcacaaaattctagcagaaggccccatgggttagaaaaggactataagaataatttcttatttaattttattttttataaaatacagaatacatacaaaagtgtgtataaaatgtacgttttaaaaaaggataagtgaacacccatgaacctactacccaggttaagaaaataaatgtcaccaggtacttgagaaacccctcgattg

SEQ ID NO: 13 - DNA, 3139 bp, Homo sapiens
acagacagaaacgagggaagagagagaaattggggaccctgaagaaagggggccagcagcaggtaggtgccatcagggacaagaacagcacctcccagggtgggagaccccaggcctttctggcagcaggtctggatggaaagtggacaggaggctcacccgtctgcatcccctgctcctgcccctgctcggctacaaaaaccaaagggacagcagctgaccacaccccggtagccactcctgcataaagctctcccctcctgtgaccagctgaggacctcaggctgcagcggagccatgcccagatacacggtgcacgtacgtggggaatggctggcagtgccctgccaggacgcgcagctcactgtgggctggctgggccgggaggccgtgaggcgctatatcaagaataagcccgacaatggtggcttcacctccgtggatgacgcgcacttccttgtgcgccggtgcaagggcctgggcctgctggacaacgaggaccggctcgaggtggccctagagaacaacgagttcgtggaagtggttatagagggtgatgccatgtctcctgacttcattccatctcaaccagaaggagtttatctatacagcaagtaccgggagcctgaaaagtacatcgagttagatggagaccgtctgaccacggaggatctggtcaacttgggaaagggacgctacaaaataaagctcaccccaacagctgagaagagggtgcagaaatccagggaggtcatagatagcatcataaaagagaaaacagttgtttacggtattactacaggttttgggaaatttgccagaactgtaattcctatcaataagctacaggagcttcaggtcaacttagtacgctcacattcttcaggtgttgggaaaccactaagtcctgagaggtgtcggatgctcttggctttaaggatcaatgtcttagccaaaggatacagtggcatttccctggagaccctcaaacaagtcatagaaatgtttaatgcctcctgcctgccctatgtcccagagaaaggaaccgttggtgccagtggagaccttgccccactctctcatcttgctcttgggctagttggagaagggaagatgtggtctccgaagagtggctgggctgatgctaaatacgtgctagaagcccatggattgaaaccagttattttaaaaccaaaagagggcctggcactcatcaatgggacgcagatgatcacatccctgggctgtgaagctgtagagcgagccagtgctattgcacggcaggctgacattgtggcagccctgacccttgaggtgctgaagggcaccaccaaagcctttgacactgacattcatgctcttcgacctcaccgtgggcaaattgaagttgcttttcggtttcggtcactcttggactcagatcaccacccatcagaaatagcagagagtcacaggttctgtgatcgcgtccaggatgcatacaccttgcgctgctgtccacaggtccatggtgtggtgaatgatacaatagcatttgtgaagaacatcattaccacagaactgaacagcgcaacagataatcctatggtctttgccaataggggagagacagtttctggaggaaacttccatggtgaatacccagccaaagccctagactacttggccattggcatccatgaacttgctgcaatcagtgagagaagaatcgagcggctctgcaatccctccctcagtgagctgcctgccttcctggtggctgaaggtggtctgaactctgggttcatgatagctcactgcacggcagcagcccttgtttctgagaacaaggctctgtgccatccctcgtctgttgactccctctccaccagcgcagccacggaggaccacgtctccatgggaggatgggcagcaaggaaagccctcagggtcatcgagcatgtggagcaagtgctggccatcgagctccttgcagcctgccagggcatagagtttctacgtcccctgaaaacaaccactccgctggagaaggtctatgacctggtgcgctctgttgtaaggccctggataaaagatcgcttcatggccccggacatcgaggcagcccacaggctgctcctggagcagaaggtttgggaagtagctgctccatacattgaaaaatacagaatggagcatattccagaatcaagacctctttctccaacagccttttcactgcaatttctgcacaagaaatccaccaaaatcccggagtctgaggacctttaatgggctttgtcatgaagtagcagatgagagggcagtcagtttagcacaaagcaatactaggctgaaggagagacctgagaactttcctaggtagatcaatccattgtatcattcagttcttctaaagcctacgttggttaggctgatggcagtattatagttgctaaattcagcactgtgttcctgttgtcgtggttcaagacccaccaggtattttcagattataaaacttttctttctttcttaacagtttcaacaggccactcactcttaagggtgagaagaataaccacaattgtatgtgcctgttttttactcttagcattagatgaattcaaatttggaaacagattgatagcaattttttctaaaaacattagacttttgttaacctttttttttttttttaaatttgcttcaacaagctctccaccagttgactttctttggctaattttactttgcatgatatgccttaatatgccttcataaataaccattttaagtcataatttgtccttaagctgcttttttcttctattaattggatcatagtaaagagtagtcaatagggtcttcagctattaattgtagaggtgattaaaaccaacaaggagtttcatgtgcaaaggagataaggaatgaatataaagattgctatttgggtggctcttattaaactgtgtattttgtacttatcactacacgtatcccccaaatgcttacatgggagtttgaggttagtattttcacttccttggtgttagtactctattcacattcttattgtaaccttcctcatttcacagataaggaatctttggggattaaccaacctcctttctgtaatggtaatcattaaaataag

(19) United States
(12) Patent Application Publication
(10) Pub. No.: US 2008/0152650 A1
(43) Pub. Date: Jun. 26, 2008

(54) Methods and Compositions For Specifically Targeting Human Hepatocellular Carcinoma Cells

(75) Inventors: Gary Rosenberg, Bothell, WA (US); Shawn P. Iadonato, Seattle, WA (US)

Correspondence Address: DAVIS WRIGHT TREMAINE, LLP/Seattle, 1201 Third Avenue, Suite 2200, SEATTLE, WA, 98101-3045, US

(73) Assignee: ILLUMIGEN BIOSCIENCES, INC., Seattle, WA (US)

(21) Appl. No.: 11/568,389

(22) PCT Filed: Apr. 27, 2005
(86) PCT No.: PCT/US05/14668

§ 371 Date: Jun. 11, 2007

Related U.S. Application Data
(60) Provisional application No. 60/565,688, filed on Apr. 27, 2004.

(51) Int. Cl. A61K 39/395; A61K 38/19; A61K 31/7088; A61K 38/45; A61K 31/7105; G01N 33/53; A61P 35/00

(52) U.S. Cl. 424/135.1; 514/12; 424/141.1; 424/130.1; 424/94.5; 514/44; 435/7.1

(57) ABSTRACT

Particular aspects of the present invention provide methods and compositions for the targeting and/or treating hepatocellular carcinoma (HCC) cells to affect cancer cell growth or viability. Exemplary methods and compositions relate to cell-associated HCC proteins (e.g., SEQ ID NOS:1-8, corresponding to PGMRCI (prostaglandin receptor membrane component 1), SEMA5A (semaphorin 5A), SLC2A2 (solute carrier family member), ABCC2 (ATP-binding cassette subfamily C member 2) and HAL (histidine ammonia lyase)), and are based, at least in part, upon the discovery that specific target genes and/or gene products are up or down-regulated in diseased tissue relative to normal tissue or in tissue of patients having other ailments. Inventive compositions comprise, for example, antibodies, antisense and siRNA agents.

### CROSS-REFERENCE TO RELATED APPLICATIONS

This application claims the benefit of priority to U.S. Provisional Patent Application Ser. No. 60/565,588, filed 27 Apr. 2004, and entitled METHODS FOR SPECIFICALLY TARGETING HUMAN HEPATOCELLULAR CARCINOMA CELLS, and which is incorporated by reference herein in its entirety.

### FIELD OF THE INVENTION

Aspects of the present invention related generally to hepatocellular carcinoma cells, and more particularly to methods and compositions for targeting and treating hepatocellular carcinoma cells, for screening for therapeutic compounds.

### BACKGROUND

Identification of molecular targets or pathways specific to the malignant cells would have substantial utility to affect the growth and viability of cancer cells without affecting non cancer cells. There is a pronounced need in the art for identification of such targets on human hepatocellular carcinoma (HCC) cells to provide methods and compositions for affecting the growth or viability of these cancer cells.

### SUMMARY OF PARTICULAR ASPECTS OF THE INVENTION

Expression microarray technology has enabled the identification of a number of genes that are expressed at significantly higher or lower levels in HCC tissue relative to non-tumor tissue. Such genes and their encoded polypeptides are the subject of particular aspects of the present invention which relates to the specific targeting of hepatocellular carcinoma cells. These molecular targets provide a means to design and create agents which will specifically alter cell processes in the cancer cells or tumors resulting in reduced cell growth or viability. These targets are such that a molecular agent or compound that is designed and created to interact specifically with the target molecule is likely to preferentially affect only those cells expressing the target molecule. A variety of such targeting agents and corresponding methodologies are described below.

The nature of these genes and their encoded polypeptide or protein products dictates the method by which they can be utilized as targets specific to cancer cells. Even though all of the encoded polypeptides of the present invention are cell associated, they can be segregated into distinct categories. Such target polypeptide categories include receptors found on the surface of the cell, including, prostaglandin receptor membrane component 1 (PGRMCI, SEQ ID NO: 1) and semaphorin 5A (SEMA5A, SEQ ID NO:2), as well as the membrane bound transporters ‘solute carrier family member’ (SLC2A2, SEQ ID NO:3) and ATP-binding cassette subfamily C member 2 (ABCC2, SEQ ID NO:4). The membrane associated target polypeptides, SEMA5A (SEQ ID NO:2), PGRMC1 (SEQ ID NO:1), ABCC2 (SEQ ID NO:4) and SLC2A2 (SEQ ID NO:3) are up-regulated in tumor tissue in comparison to non-tumor tissue. These proteins can be targeted by naked antibodies, antibody-based reagents, or antibodies or antibody-based reagents conjugated or coupled to compounds that alter cell function. A diverse array of such compounds may be employed in the methods of the present invention, including proteins, toxins or cytotoxic agents, and radioisotopes.

The membrane associated target polypeptides of the present invention can also be targeted by antagonists (e.g., for SEQ ID NOS:1-2) or inhibitors (e.g., for SEQ ID NOS:3-4). Alternatively, receptor function associated with SEMA5A (SEQ ID NO:2) and PGRMC1 (SEQ ID NO: 1) can be affected by compounds or agents that bind the corresponding receptor's ligand. Such compounds useful in the methods of the present invention include anti-ligand antibodies and soluble forms of the receptor.

Additionally, the expression of the up-regulated polynucleotides SEMA5A (SEQ ID NO:2), PGRMC1 (SEQ ID NO:1), ABCC2 (SEQ ID NO:4), and SLC2A2 (SEQ ID NO:3), can be inhibited by antisense technology (and including siRNA methods). This is established technology in which polynucleotides, including genomic DNA, cDNA, RNA, siRNA, ribozymes, and derivatives such as S-oligonucleotides, complementary to the polynucleotide sequences of interest, are administered to inhibit expression of genes encoding the target polypeptides.

A fifth target polypeptide of the present invention is a cytoplasmic enzyme, histidine ammonia lyase (HAL, SEQ ID NO:8). Expression of the gene encoding HAL (SEQ ID NO:8) is down-regulated in tumor tissue as compared to non-tumor tissue. The decrease in HAL (SEQ ID NO:8) gene expression in tumor tissue indicates that increasing the expression of HAL, or its corresponding polypeptide, will detrimentally affect HCC cell growth or viability. The present invention includes gene therapy approaches aimed at increasing HAL (SEQ ID NO:8) activity by administration of a polynucleotide encoding HAL (SEQ ID NO:8). Similarly, the HAL target polypeptide (SEQ ID NO:8), or an active fragment thereof, can be administered. Additionally, down regulation of this enzyme in disease tissue is expected to result in increased levels of histidine and histamine and decreased levels of urocanic acid providing additional approaches to selectively targeting HCC cells.

The discussion below is descriptive, illustrative and exemplary and is not to be taken as limiting the scope of any inventive defined by any presently or subsequently appended claims.

### DETAILED DESCRIPTION

The term “treating” as used herein is intended to encompass treating, preventing, curing or ameliorating a condition (e.g., hepatocellular carcinoma) in a patient having or at risk for the condition.

In particular aspects, expression microarray analysis of tumor samples from Hepatitis C (HCV) infected patients with hepatocellular carcinoma (HCC) led to the identification of genes that were specifically up or down-regulated in hepatocellular carcinoma tumor tissue when compared to HCV infected, cirrhotic non-tumor tissue, and normal liver tissue.

Liver and HCC samples were obtained during surgical procedures with prior informed consent from all persons involved. HCC samples included 21 from HCV infected patients and 1 from a patient infected with Hepatitis B. In addition, 4 samples of normal, non-diseased liver and 8 samples of HCV infected, cirrhotic liver with no evidence of HCC were used for analysis. Total RNA was isolated as described in Geiss et al. (2001). RNA amplification was performed using a T7 RNA polymerase protocol (Eberwine, 1996) with the AmpliScribe™ Transcription kit (Epicentre Technologies, Madison, Wis.) as described by the manufacturer. The quality of amplified RNA samples was evaluated using capillary electrophoresis in an Agilent 2100 Bioanalyzer (Agilent Technologies, Palo Alto, Calif.).

cDNA microarrays were constructed by the University of Washington's Center for Expression Array Technology using PCR products generated by amplification of sequence verified I.M.A.G.E. consortium clones obtained from Research Genetics (St. Louis, Mo.) (Lennon et al. 1996). Microarrays were constructed as previously described (Geiss et al. 2001). A human high density set consisted of two arrays, each of which represented 7,296 human clones in duplicate with a number of additional control sequences, for a total of 14,976 clones (approximately 13,597 unique I.M.A.G.E. cDNA clones). Each single experiment involved interrogation of two slides for which dye labels had been reversed (fluor reversal methodology as described in Geiss et al. 2000; Geiss et al. 2001). A total of at least four separate hybridization measurements were taken per gene per experiment. Protocols used for probe synthesis, microarray hybridization, and wash conditions were as previously described (Geiss et al. 2001).

Microarrays were scanned and the images were quantified using a custom spot-finding program, Spot-On Image (Geiss et al. 2000 and Geiss et al. 2001), that calculated the standard deviations and the mean ratios between the expression levels of each gene in the analyzed pair of samples. Raw data and sample information were entered into a custom designed database, Expression Array Manager, and evaluated using Rosetta Biosoftware's Resolver® Version 3.0 (Rosetta Biosoftware, Kirkland, Wash.), a software package for the storage and analysis of microarray expression data. This package implements common statistical procedures (clustering, trend analysis, similarity searches based on a BLAST-related algorithm, etc.) together with a sophisticated error model to compensate for biological and experimental variation.

The expression microarray data was processed by examining only HCV-infected HCC patient samples and sorting for genes that were significantly (p<0.01) up or down-regulated (more than two-fold) in tumor versus non-tumor liver samples from the same patient. Genes that met these criteria in eight or more patients were then analyzed in control samples from HCV infected patients with liver cirrhosis but no tumors and also in samples of normal healthy liver. If the expression of the gene was unchanged or changed in the opposite direction in control samples, its potential for use as a therapeutic target was further evaluated using information available in the National Center for Biotechnology Information databases (Unigene, OMIM, LocusLink, and HomoloGene) and currently published literature regarding the location and function of its polypeptide product.

Target polypeptides of the present invention comprise protein products of genes that are preferentially or specifically up-regulated or down-regulated in HCC tissue. Such polypeptide, and genes and RNA encoding them are viable pharmacological or therapeutic targets for the treatment of HCC due to their location or activity and include PGRMC1 (SEQ ID NO:1), SEMA5A (SEQ ID NO:2), ABCC2 (SEQ ID NO:4), SLC2A2 (SEQ ID NO:3) and HAL (SEQ ID NO:8). The amino acid sequences of the target polypeptides and certain variants thereof are listed herein in the Sequence Listing (e.g., SEQ ID NOS:1-8). The differential expression of these genes provides for a number of ways to specifically target HCC cells in order to affect their growth and or viability. These methodologies are the subject of particular aspects of the present invention and are detailed below.

### Description of the Target Polypeptides of SEQ ID NOS: 1-4

PGRMC1 (SEQ ID NO:1). PGRMC1 is a progesterone receptor. While many progesterone receptors are intracellular, PGRMC1 is believed to be localized to the plasma membrane (Krebs et al. 2000). The activity of progesterone receptors is dependent upon progesterone binding which is followed by a translocation of the receptor to the cell nucleus.

SEMA5A (SEQ ID NO: 2). The semaphorin family comprises a large number of secreted and membrane bound members. The neutropilins and the plexins serve as semaphorin receptors. SEMA5A is a membrane bound protein. Neutropilin and/or plexin are believed to be ligands for SEMA5A (Adams and Tucker 2000). The neutropilins and plexins are membrane bound, suggesting that SEMA5A binding with these molecules results in a cell to cell interaction. Alternatively, SEMA5A may bind an as yet unidentified molecule such as a soluble form of a plexin or neutropilin (see examples below).

SLC2A2 (SEQ ID NO:3). SLC2C2 is a facilitative glucose transporter. It belongs to a family of 12 transmembrane domain proteins. Binding extracellular glucose results in a transformational change that relocates glucose into the cell (Oka et al. 1990).

ABCC2 (SEQ ID NOS:4-7). ABCC2 is an integral membrane protein involved in multi-drug resistance. It functions in the energy-dependent transport of chemotherapeutic agents and other molecules out of hepatocytes (Gerk and Vore 2002).

### Targeting of Cancer Cells

In particular aspects, the identified targets provide at least two approaches by which the growth and or viability of the HCC cells may be effected. The surface receptors (SEQ ID NOS:1-7) can simply be used as specific targets without regard to the biology of these molecules. An agent that specifically binds a surface receptor can be used to deliver a locally-acting biological agent (e.g., therapeutic agent) that will affect the targeted cell. The nature of the targeted molecule is important only in that it is accessible to the targeting agent and that it is found in significantly greater concentrations on the cancer cell than non-cancer cells. For example, an antibody-radioisotope conjugate that binds a membrane receptor present exclusively on HCC cells would be expected to affect only those cells expressing the receptor (HCC cells). Alternatively, immunization of an individual with a target molecule, or derivantive thereof, may prompt the individual's immune system to mount an immune response specific to the target molecule resulting in elimination of those cells expressing said molecule.

In additional aspects, another approach to the utilization of the target molecules is based on a presumed causal relationship between the observed change in their expression in tumor cells, and cancer cell growth or survival. Interfering with the expression or biological function of molecules up-regulated in tumors would be expected in such instances to be detrimental to cell growth or viability. Those targets that are down-regulated in tumors may interfere with growth or viability and therefore up-regulation or replacement of their function would be expected to reduce growth or viability of the specific cells involved.

Examples of particular approaches in the utilization of the identified target molecules are noted below.

### Antibodies Useful in the Methods of the Present Invention

The term “antibody” is used in the context of this invention to include a variety of molecules familiar to one skilled in the art. Antibodies provide a means to specifically target cells at a molecular level by binding specific molecules or antigens. In the present invention, the molecules targeted by antibodies are the polypeptides or fragments of the polypeptides as defined by SEQ ID NOS:1-7. Antibodies, and/or antibody-based reagents specific to these molecules can be generated by a variety of methods and can exist in a variety of forms as described below.

Antibodies can be polyclonal, monoclonal, single chain Fv, recombinant chimeric molecules, and fragments such as Fab′, Fab′(2), minibodies, and domain deleted antibodies. Antibodies are identified and produced by a variety of means including, but not limited to: in vivo production in rabbits, sheep, rats, mice; production of recombinant molecules in vitro in mammalian, fungal, bacterial, insect or plant cells or in transgenic animals; selection in phage display or recombinant yeast systems; and chemical or proteolytic modification of any of the molecules noted above. A description of these antibodies and their selection and production is found in the following references: King et al. 1994; Xiang et al. 1997; Glennie and Johnson 2000; Green 2000; Nuttall et al. 2000; Huston and George 2001; Kriangkum et al. 2001; Reff and Heard 2001; Siegel 2002.

Antibodies and antibody conjugates which target molecules specific to cancer cells or other molecular targets, are useful as they can specifically alter the growth or the viability of only those cells expressing the target molecule. However, antibodies as in vivo therapeutics present several difficulties. Antibodies of non-human origin may induce a host immune response. Another problem is that antibodies often do not penetrate tumors well due in part to their size. To overcome these problems, a variety of approaches have been taken and are well documented in the literature (Reff and Heard 2000; Reiter 2001). For example, to render non-human antibodies less antigenic, molecular biological approaches have been taken to replace non-human regions of the antibody with equivalent regions from human immunoglobulins while leaving the complementarity regions intact (Morrison et al. 1984; Reff and Heard 2001). These techniques range from, simple substitution of the non-human constant regions of the antibody with the constant regions of human immunoglobulin molecules, to more sophisticated methodologies where the non human complementarity regions on the non human immunoglobulin are spliced, grafted, or engineered into a human immunoglobulin molecule (Jones et al. 1986). An important example of this technology is Herceptin® (trastuzumab) which is a humanized mouse monoclonal antibody used to treat breast cancer (Carteret al. 1992; Goldenberg 1999).

Another example, Rituxan® is used to treat non-Hodgkins lymphoma and consists of a murine variable region fused to a human gamma-1 constant region (Johnson and Glennie 2001; Maloney et al. 2002).

Another type of antibody useful in the practice of the present invention is a Primatized® antibody. Primatized® antibodies are developed by immunizing cynomologous monkeys. The antibody variable regions of the cynomologous antibodies are indistinguishable from the homologous human molecule. As is the case with Rituxan®, human immunoglobulin constant regions are spliced onto the cynomologous variable region. Primatized® antibodies have been developed to treat lupus and allergic asthma (Newman et al. 1992; Nakamura et al. 2000).

Aspect of the present invention also include the use of human antibodies obtained from transgenic animals (Green 1999). These antibodies are identified and characterized in the same manner as those from non-transgenic animals but would not illicit the immune response normally expected with nonhuman antibody therapeutics. Human antibodies have been generated in mice against several therapeutic targets including interleukin-8 (Yang et al. 1999), and epidermal growth factor (Davis et al. 1999; Yang et al. 2001).

Antibodies can also be chemically modified to render them less antigenic, thereby improving the pharmacokinetic properties for use in vivo. The most commonly used technique is to covalently bind polyethylene glycol to the immunoglobulin molecule (Chapman 2002). This has been done without loss of efficacy with a monoclonal anti-interleukin-8 antibody used to prevent edema in ischemia reperfusion injury (Leong et al. 2001) and with a monoclonal antibody used to treat colon cancer (Deckert et al. 2000). Antibody fragments have also been used in vivo to affect cell growth or viability and offer several advantages. Removal of portions of the antibody molecule may render it less immunogenic and increase half-life in circulation. Their reduced size allows more rapid diffusion, thereby enhancing the ability to penetrate solid tumors. There are a variety of antibody fragments which have been generated in a number of ways. Such fragments include single chain Fv, Fab′ and Fab′(2) and chimeric versions thereof (Behr et al. 1995; Glennie and Johnson 2000; Kortt 2001; Weir et al. 2002), minibodies (Tramontano et al. 1994; Hu et al. 1996), and domain deleted antibodies (Reff and Heard 2001), all of which have been reviewed in the literature in terms of development, selection and production (Reff and Heard 2001).

Phage display technology has enabled the selection of single chain antibodies from libraries of human immunoglobulins (Dani 2001; Rhyner et al. 2002). As an example, an anti-carcinoembryonic antibody for the treatment of cancer has been isolated from a phage scfv library (Chester et al. 2000). An embodiment of the present invention features the use of single chain antibodies to block ligand binding of the polypeptides of SEQ ID NOS:1-2, thereby affecting the viability or growth of HCC cells.

### Inhibition of the Biological Activitv of the Target Polypeptides of SEQ ID NOS: 1-7

Antibodies that bind receptors and block ligand binding without receptor activation (antagonists) are a means to specifically target and impact the biological activity of cells expressing those receptors. Antibodies that specifically bind the target polypeptides of SEQ ID NOS:1-7 or fragments thereof form a part of the present invention. One skilled in the art is capable of producing said antibodies or in the case of recombinant antibody libraries, screening for said antibodies.

For example, rabbits or mice or other suitable animals are immunized with peptide fragments of PGRMC1 (SEQ ID NO:1), which are from regions at or near the progesterone binding site. Some of the antibodies generated in this way are expected to bind PGRMC1 and sterically interfere with progesterone binding preventing receptor activation by progesterone. Analogous antibodies for each of the other membrane associated polypeptides of SEQ ID NOS:2-7 can be obtained similarly. Similar approaches are known in the art. Anti-peptide antagonists have been generated, for example, which inhibit the biological activity of interleukin-1 accessory protein (Yoon and Dinarello 1998) and epidermal growth factor receptor (Gentry and Lawton 1986).

An activity assay can be used to identify antibodies with therapeutic potential. Said assay would consist of screening a single chain Fv (scfv) phage display library on a cell based assay. As an example, an scfv phage display antibody library is first screened verses SLC2A2 (SEQ ID NO:3) or peptide fragments thereof. Single chain antibodies would be cloned from SLC2A2 reacting phage and further tested on SLC2A2 transformed oocytes as developed by Permutt et al. (1989) that have SLC2A2 glucose transporter activity. Those scfvs that inhibit SLC2A2 activity have therapeutic potential. In this case, the functional assay is of low throughput so the primary screen consists of identifying those phage expressing scfvs that bind the target polypeptide. In other instances, a high throughput activity assay may be available, obviating the need for a binding assay as a primary screen (see the next example).

As a third example, monoclonal antibodies generated against PGRMC1 (SEQ ID NO:1) are amenable to use in an MDCK cell assay which measures export of radio-labeled dinitrophenyl GSH (Evers et al. 1998). Antibodies that block the efflux of the radio-labeled compound have therapeutic potential in the treatment of HCC. This assay is relatively high throughput so that antibodies of therapeutic potential can be identified without a second screen. Antibody antagonists have been produced against a number of previously identified human cell surface receptors including epidermal growth factor receptor (Crombet-Ramos et al. 2002) and interleukin-2 receptor (Olive et al. 1986).

In another embodiment of the present invention, an antibody binding to a receptor inhibits receptor function without inhibiting ligand binding. Ligand binding normally will induce a structural change in the receptor leading to signal transduction, subunit dissociation, internalization, or some combination thereof with which the binding of an antibody to the receptor may interfere. Antibodies generated against one or more of the polypeptides of SEQ ID NOS:1-7, in this aspect of the present invention, are screened for the ability to block receptor function. Some of the antibodies testing positive in such a screen would be competitive inhibitors of ligand binding while others would be expected to inhibit receptor function without grossly affecting ligand binding.

Certain receptors have both stimulatory and regulatory ligands. Another embodiment of the present invention therefore includes the use of inhibitory ligands including growth factors, cytokines, chemokines, and other naturally occurring molecules that bind the polypeptides encoded by SEQ ID NOS: 1-7 and block their respective activities. These molecules are identified using assays based on ligand binding or ligand induced receptor activation. Compounds are screened to identify those that block ligand binding or reduce ligand induced activation of the receptor. Sources of inhibitory ligands include, but are not limited to, conditioned medium from cultured mammalian cells, synovial fluid, serum, plasma, spinal fluid, and the like.

Small molecule receptor inhibitors have been isolated by high throughput screening of compounds (Landro et al. 2000). The source of these compounds varies but includes collections of natural molecules (Munro et al. 1999; Harvey 1999), combinatorial chemical libraries (Floyd et al. 1999; Ramstrom and Lehn 2002), or synthetic peptide libraries (Shusta et al. 1999). Particular aspects of the present invention include molecules that specifically bind and inhibit activation of the polypeptides of SEQ ID NOS:1-7 to be used in targeting HCC cells. Examples of screening assays for the identification of such small molecule inhibitors are described above.

### Patient Immunization as a Means to Develop Inhibitory Antibodies

Patients may be immunized with one or more of the target polypeptides SEQ ID NOS: 1-7 or immunogenic fragments thereof in order to induce an immune response. This will induce the patient's immune system to preferentially destroy the tumor cells expressing these polypeptides. The literature contains a number of like examples including immunization by antiidiotypic antibodies for the treatment of melanoma (Lutzky et al. 2002), immunization with melanoma antigens for the treatment of the disease (Perales and Wolchok 2002) and immunization with recombinant fusion protein containing portions of the human epidermal growth factor receptor (Vidocvic et al. 2002).

### Receptor Ligands as Targets

To inhibit the activation or activity of receptors encoded by SEQ ID NOS:1-2, ligands are targeted to prevent them from binding their respective receptors. Binding ligands can be accomplished in a variety of ways as noted below, which are embodied in the present invention as a means to target HCC cells affecting growth or viability.

Genes encoding soluble receptors based on the target polypeptides SEQ ID NOS: 1-2, are predicted and produced using standard molecular biological techniques. These molecules contain at least the ligand binding portion of the respective receptor and may or may not include a portion of the membrane associated part of the molecule. This concept is illustrated by the rheumatoid arthritis drug, Enbrel® which binds TNF and prevents the ligand from binding and activating the TNF-receptor. Enbrel® is a chimeric molecule which is a fragment of an immunoglobulin molecule combined with the ligand binding region of the TNF receptor that is produced recombinantly in mammalian cells (Murray and Dahl 1997).

Some receptors exist in 2 forms, one being membrane bound and the other soluble. For example, the receptors for TNF-alpha and interleukin-1 exist in membrane and soluble forms. The soluble forms were developed as therapeutics for inflammation and sepsis (Lowry 1993; Kluth and Rees 1996). A similar inhibitor based on the sequence of the target polypeptides of the present invention (SEQ ID NOS:1-2) or on naturally occurring soluble receptors for the ligands of PGRMC1 or SEMA5A is an embodiment of the present invention.

Another way to bind ligands and render them unavailable for receptor activation is to administer a ligand specific antibody. In another embodiment of the present invention, antibodies that bind the ligands of the target polypeptides (SEQ ID NOS:1-7) are employed. This approach has been successfully employed in targeting cancer cells that over express the epidermal growth factor receptor (Yang et at. 2001). Anti-epidermal growth factor ligand antibodies were shown to inhibit tumor cell proliferation and eradicate tumors in a mouse cancer model.

### Antibody Conjugates and Immunotoxins

Each of the target polypeptides SEQ ID NO:1-7 are expressed on the surface of HCC cells and are accessible to exogenous molecules. As these target polypeptides are present at higher levels on HCC cells as compared to non-cancer cells, they can be utilized as preferential targets for systemic antibody-based therapies. The differential expression of these target molecules enables the specificity of antibody-based therapy meaning that cytotoxic antibodies directed against the target polypeptides SEQ ID NOS: 1-7, preferentially affect HCC cells over normal tissue. Therefore, the present invention includes antibodies specific to one or more of the target polynucleotides of SEQ ID NOS: 1-7 that will enable or facilitate treatment of HCC.

Antibody therapies are well described in the literature and involve several distinct approaches. These include, but are not limited to, naked antibodies, antibodies conjugated or coupled to toxins or other biologically active compounds (immunotoxins), radioimmuno conjugates (radionuclide antibody), and antibody coated liposomes which contain one or more biologically active compounds.

Binding of an antibody to a cell in itself is sometimes enough to inhibit growth (cytostatic effect) or kill the target cell (cytotoxic effect) (Baselga et al. 1998; Czuczman et al. 1999). The mechanism of this activity varies but may involve antibody-dependent cell mediated cytotoxicity (Clynes et al. 2000), activation of apoptosis (Maloney 2001), inhibition of ligand-receptor function, or a signal for complement fixation. In fact it has been suggested that anti-cancer chimeric antibody rituximab, owes its potency to the fact that it exhibits several of the activities noted above (Maloney 2001; Park and Smolen 2001). Some antibodies are cytostatic, not cytotoxic. For example, trastuzumab, which is a well characterized anti-HER2 antibody and is an effective anti-cancer agent, is, at least in vitro, cytostatic. The present invention pertains to antibodies which specifically bind to target polypeptides SEQ ID NOS: 1-7 and are either cytoxic or cytostatic.

Antibodies can also be conjugated or coupled to a diverse array of compounds which include, but are not limited to proteins, toxins or cytotoxic agents, radionuclides, apoptotic factors (Wuest et al. 2002), anti-angiogenic compounds or other biologically active compounds which will inhibit the growth of or kill the target cell or tissue. For example, cytotoxic or cytostatic agents include, but are not limited to, diphtheria toxin (Kreitman 2001 a), Pseudomonas exotoxin (Kreitman 2001 a; Kreitman 2001 b), ricin (Kreitman 2001 a), gelonin, doxorubicin (Ajani et al. 2000) and its derivatives, iodine-131, yttrium-90 (Witzig 2001), indium-111 (Witzig 2001), RNAse (Newton and Ryback 2001), calicheamicin (Bernstein 2000), apoptotic agents, and antiangiogenic agents (Frankel et al. 2000; Brinkmann et al. 2001; Garnett 2001). These have been all shown to adversely affect cells targeted by antibodies specific to targeted cell antigens.

Toxins can also be targeted to specific cells by incorporation of the toxin into antibody coated liposomes. The antibody directs the liposome to the target cell where the bioactive compound is released. For example, cytotoxins in antibody coated liposomes have been used to treat teratocancinoma (Marty et al. 2002) and BER2 expressing xenografts (Park et al. 2002) in animal models. These targeted liposomes can also be loaded with DNA encoding bioactive polypeptides such as inducible nitric oxide synthase (Khare et al. 2001).

Prodrugs or enzymes can also be delivered to targeted cells by specific antibodies. In this case the immunoconjugate consists of an antibody coupled to a drug that can be activated once the antibody binds the target cell. Examples of this strategy have been reviewed (Denny 2001; Xu and McLeod 2001). Antibody-prodrug/enzyme conjugates targeted to the polypeptides encoded by SEQ ID NOS:1-7 for the treatment of HCC are an embodiment of the present invention.

The specificity and high affinity of antibody molecules makes them ideal candidates for delivery toxic agents to a specific subset of cellular targets. As the target polypeptides of SEQ ID NOS: 1-7 are present at higher levels on HCC cells than on non tumor cells, they provide excellent targets for antibody-based therapies.

### Antisense

The genes encoding the target polypeptides of SEQ ID NOS: 1-7 are themselves targets for antisense therapy which will inhibit expression of these genes. These methods constitute an embodiment of the present invention and consist of delivery of polynucleotides, either DNA, RNA, ribozymes, peptide nucleic acids, or non-nucleic acid polymers such as phosphorothionate or morpholino derivatives that specifically bind DNA or RNA in a base pair dependent manner. Design, production and characterization of these agents have been reviewed in the literature (lyer et al. 1990; Cohen 1994; Agrawal and lyer 1997; Merdan et al. 2002). Antisense molecules are complimentary to the polynucleotide sequences or genes encoding the target polypeptides of SEQ ID NOS: 1-7 and will inhibit the corresponding RNA or protein synthesis of such genes. The complimentary polynucleotide or related molecule is preferably of sufficient length to hybridize specifically to at least ten contiguous nucleic acids encoding one of the target polypeptides of SEQ ID NOS:1-7.

HAL (SEQ ID NO: 5). Aspects of the present invention also pertain to the gene encoding the target polypeptide HAL (SEQ ID NO8) that is down regulated in HCC tissue, making this target polypeptide amenable to gene therapy. Gene therapy includes replacement of the gene by delivery of a polynucleotide, either DNA or RNA, that encodes a polypeptide that is at least 88% identical to the HAL target polypeptide SEQ ID NO:8. Gene therapy targeted to the liver has been extensively reviewed both in terms of delivery and vector choices (Guha et al. 2001; Mazzolini et al. 2001; Schmitz et al. 2002; Wu et al. 2002). HAL (SEQ ID NO:8) may also be replaced directly. In another embodiment of the present invention, the target polypeptide of SEQ ID NO:8 is administered to treat HCC. This embodiment includes HAL (SEQ ID NO:8) and HAL-related polypeptides including fragments of the polypeptide that have the biological activity of the full-length, native HAL molecule as described below. HAL (SEQ ID NO:8) is the first enzyme in histidine and histamine catabolism (Suchi et al. 1995). Decreased levels of this enzyme may result in increased levels of histidine and histamine and decreased levels of urocanic acid, the product of HAL (SEQ ID NO:8) catalysis. The fact that HCC cells produce reduced levels of HAL (SEQ ID NO:8) indicates that histidine or histamine are required for cancer cell survival or proliferation and/or urocanic acid or other mol ecules derived from histidine and histamine inhibit cancer cell survival or growth. This aspect of the present invention therefore includes the use of urocanic acid and other histidine and histamine catabolites including 4imidazalone-5-propionic acid and N-formimino-glutamic acid alone or in combination with each other or HAL (SEQ ID NO:8) in the treatment of HCC.

### Antihistamines

Increased levels of histamine in HCC patients may affect cancer cell survival or proliferation. Indeed, one of the histamine receptors, H2, has been implicated in regulation of cell growth (Suh et al. 2001). Therefore, another embodiment of the present invention includes the use of histamine antagonists in the treatment of HCC. Histamine antagonists constitute a diverse array of compounds which have been extensively reviewed (Greaves 2001; Walsh et al. 2001).

### Combination Therapy

Cancer is often effectively treated by a combination of reagents or methodologies. The growth or viability of HCC cells may also be affected by treatment with a combination of agents or methodologies. Examples include:

1) chemotherapy and radiation therapy in the treatment of cervical cancer (Aoki and Tanaka 2002) or head and neck cancer (Busto et al. 2001) or pancreatic cancer (McGinn et al. 2002);

2) chemotherapy and surgery in the treatment of cervical cancer (Aoki and Tanaka 2002);

3) antibody therapy and cytokine therapy in the treatment of breast cancer (Hortobagyi 2002);

4) combination chemotherapy treatment of melanoma (McClay 2002) or colorectal carcinoma (Kim et al. 2002);

5) the suggestion of multiple therapies including gene therapy, angiogenesis inhibitors and antibody therapy in the treatment of non-small cell lung cancer (Felip and Rossell 2001); and

6) the suggested treatment of metastatic breast cancer by a combination of chemotherapy and antibody or kinase inhibitor, or angiogenic inhibitor therapy.

Thus, the therapeutic agents and constructs of the present invention are contemplated for use in combination with one or more standard cancer treatments. For example, particular inventive methods may be used in combination with one or more of the following:

a) a chemotherapeutic agent;

b) radiation therapy;

c) surgical resection or liver transplantation; or

d) radio frequency ablation, cryosurgery, ethanol ablation and embolization.

### Prophylactic Treatment of HCC

Current diagnostic methods for HCC are unable to reliably detect the cancer at its earliest stages. In patients at high risk for HCC, prophylactic administration of a therapeutic molecule of the present invention may be appropriate. Patients at high risk for HCC are those with chronic liver disease including hepatitis B and C patients, and those with cirrhosis of the liver (Bruix et al. 2001; Befeler and Bisceglie 2002). Thus, if a patient exhibits such increased risk of developing HCC, the targeting agents or constructs of the present invention can be administered to such at risk patients on a prophylactic basis.

### Polypeptides SEQ ID NOS: 1-7 as Discovery Tools for HCC Therapeutics

The polypeptides of SEQ ID NOS: 1-7 can be used to assay and/or screen for compounds effective in the treatment of HCC. Exemplary binding and biological function assays have been described above. Preferred modes are described here. For example, cells that do not express PGMRC 1 (SEQ ID NO:1) are transfected with the gene encoding that target polypeptide. Test agents are then screened for binding to the transfected cells but not the untransfected parent cells. Said screening is accomplished using a functional, binding, competitive, or reporter assay. Alternatively, subcellular fractions of the transfected cells are isolated and used in competitive binding or a direct binding assay. For example, radiolabeled progesterone are added to the transfected cells followed by a test compound. Test compounds that displace the radiolabeled progesterone are therapeutic candidates for the treatment of HCC as antagonists of the PGMRC1 receptor (SEQ ID NO:1).

### Particular Inventive Modulators, Compositions, Utilities and Expression Vectors

Modulators of gene expression. Particular embodiments provide modulators of cellular gene expression. Preferably, inventive modulators are directed to one or more of the cellular gene targets described herein (e.g., SEQ ID NOS:9-12) (e.g., those encoding for SEQ ID NOS:1-7), the expression of which is required, at least to some extent, for hepatocellular carcinoma.

Inventive modulators include, but are not limited to, antisense molecules, siRNA, ribozymes, antibodies or antibody fragments, proteins or polypeptides as well as small molecules. Particular modulators, such as gene-specific antisense, siRNA, and ribozyme molecules, small molecules, and antibodies and epitope-binding fragments thereof, are inhibitors of target gene expression, or of the biological activity of proteins encoded thereby.

Preferably, inventive antisense molecules are oligonucleotides of about 10 to 35 nucleotides in length that are targeted to a nucleic acid molecule corresponding to a target gene sequence, wherein the antisense molecule inhibits the expression of at least one target gene sequence (e.g., SEQ ID NOS:9-12) (e.g., those encoding for SEQ ID NOS:1-7). Antisense compounds useful to practice the invention include oligonucleotides containing art-recognized modified backbones or non-natural internucleoside linkages, modified sugar moieties, or modified nucleobases.

Preferred antisense molecules or the complements thereof comprise at least 10, at least 15, at least 17, at least 20, at lease 22, or at least 25, and preferably less than about 35 consecutive complementary nucleotides of, or hybridize under stringent or highly stringent conditions to at least one of the nucleic acid sequences encoding a polypeptide of the group consisting of: (e.g., SEQ ID NOS:1-7). Preferably, such antisense molecules are PMO (phosphorodiamidate morpholino Oligomers) antisense molecules.

Thus, the present invention includes nucleic acids that hybridize under stringent hybridization conditions, as defined below, to all or a portion of the target cellular gene sequences. The hybridizing portion of the hybridizing nucleic acids is typically at least 10, at least 15, at least 17, at least 20, at least 22, at least 25, at least 30 or at least 35 nucleotides in length. Preferably, the hybridizing portion of the hybridizing nucleic acid is at least 80%, at least 90%, at least 95%, at least 98%, or at least 99% identical to a target sequence, or to the complements thereof.

Hybridizing nucleic acids of the type described herein can be used, for example, as an inventive therapeutic modulator of target gene expression, a cloning probe, a primer (e.g., a PCR primer), or a diagnostic and/or prognostic probe or primer. Preferably, hybridization of the oligonucleotide probe to a nucleic acid sample is performed under stringent conditions. Nucleic acid duplex or hybrid stability is expressed as the melting temperature or Tm, which is the temperature at which a probe dissociates from a target DNA. This melting temperature is used to define the required stringency conditions.

For sequences that are related and substantially identical to the probe, rather than identical, it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having>95% identity with the probe are sought, the final wash temperature is decreased by 5° C.). In practice, the change in Tm can be between 0.5° C. and 1.5° C. per 1% mismatch.

Stringent conditions, as defined herein, involve hybridizing at 68° C. in 5× SSC/5× Denhardt's solution/1.0% SDS, and washing in 0.2× SSC/0.1% SDS at room temperature, or involve the art-recognized equivalent thereof. Moderately stringent conditions, as defined herein, involve including washing in 3× SSC at 42° C., or the art-recognized equivalent thereof. The parameters of salt concentration and temperature can be varied to achieve the optimal level of identity between the probe and the target nucleic acid. Guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

Antisense molecules preferably comprise at least 17 or at least 20, or at least 25, and preferably less than about 35 consecutive complementary nucleotides of, or hybridize under stringent conditions to at least one of the nucleic acid sequences encoding a target polypeptide (e.g., encoding SEQ ID NOS:1-7). Preferably, such antisense molecules are PMO antisense molecules.

The invention further provides a ribozyme capable of specifically cleaving at least one RNA encoding for a target protein (e.g., SEQ ID NOS:9-12) (e.g., those encoding for SEQ ID NOS:1-7), and a pharmaceutical composition comprising the ribozyme.

The invention also provides small molecule modulators of target gene expression, wherein particular modulators are inhibitors capable of reducing the expression of at least one target gene, reducing or preventing the expression of mRNA from at least one target gene, or reducing the biological activity of at least one target gene product. Preferably, the target gene is selected from the group encoding for a target polypeptide (e.g., SEQ ID NOS:1-7).

Compositions. Further embodiments provide compositions that comprise one or more modulators of target gene expression (or modulators of biological activity of target gene products) in a pharmaceutically acceptable carrier, diluent or excipient.

Particular embodiments provide a pharmaceutical composition for inhibiting target gene expression, comprising an antisense oligonucleotide according to the invention in a mixture with a pharmaceutically acceptable carrier or diluent.

Further provided is a composition comprising a therapeutically effective amount of an inhibitor of a target gene product (e.g., protein) in a pharmaceutically acceptable carrier. In certain embodiments, the composition comprises two or more target gene product inhibitors. Preferably, the target gene product is selected from: the nucleic acid group consisting of SEQ ID NOS:1-7 and combinations thereof.

In particular composition embodiments, the target gene inhibitor is an antisense molecule, and in specific embodiments the antisense molecule or the complement thereof comprises at least 10, 15, 17, 20 or 25 consecutive nucleic acids of, or hybridizes under stringent conditions to at least one of the nucleic acid sequences encoding a target polypeptide (e.g., SEQ ID NOS:1-7). Preferably, such antisense molecules are PMO antisense molecules.

Methods and uses. Particular embodiments of the present invention provide methods of modulating target gene expression or biological activity of target gene products in HCC cells.

The invention provides a method of inhibiting the expression of target cellular genes in human cells or tissues comprising contacting the cells or tissues in vivo (also ex vivo, or in vitro) with an antisense compound or a ribozyme of about 10 to 35 nucleotides in length targeted to a nucleic acid molecule encoding a target gene product so that expression of the target gene product is inhibited. Preferably, the target gene is selected from the group consisting of: SEQ ID NOS:1-7.

The invention additionally provides a method of modulating target gene expression in cells comprising contacting the cells in vivo (also ex vivo, or in vitro) with an inventive antisense compound or ribozyme of about 10 to 35 nucleotides in length targeted to a nucleic acid molecule encoding a target gene product so that expression of the target gene product is inhibited.

The invention provides for the use of a modulator of target gene expression according to the invention to prepare a medicament for modulating target gene expression or activity.

Additional embodiments provide a method of inhibiting target gene expression or encoded biological activity in a mammalian cell, comprising administering to the cell an inhibitor of target gene expression (or of encoded biological activity), and in a specific embodiment of the method, the inhibitor is a target gene-specific antisense molecule. Preferably, the antisense molecule is a PMO antisense molecule.

The invention also provides a method of target gene expression in a subject, comprising administering to said subject, in a pharmaceutically effective vehicle, an amount of an antisense oligonucleotide which is effective to specifically hybridize to all or part of a selected target nucleic acid sequence derived from target gene. Preferably the antisense oligonucleotides are PMO antisense compounds.

The invention further provides a method of treating HCC-realated conditions or disease, comprising administering to a mammalian cell a modulator of target gene (e.g., encoding SEQ ID NOS:1-7) expression such that, for example, the neoplastic condition or a virus-related disease is reduced in severity.

As discussed in the EXAMPLES herein below, additional embodiments provide screening assays for identification of compounds useful to modulate target gene expression (activity), comprising: contacting cells with a test agent; measuring, using a suitable assay, expression of at least one target cellular gene sequence; and determining whether the test agent inhibits said gene expression relative to control cells not contacted with the test agent, whereby agents that inhibit said gene expression are identified as compounds useful to modulate target gene or gene product activity.

Preferably, expression of at least one target cellular gene sequence is expression of respective mRNA, or expression of the protein encoded thereby.

Preferably, agents that inhibit or modulate said target gene expression are further tested for the ability to modulate HCC, or HCC-related conditions or diseases.

Further embodiments provide diagnostic or prognostic assays for HCC, maturation or progression, comprising: obtaining a cell sample from a subject suspected of having HCC; measuring expression of at least one target gene sequence; and determining whether expression of the at least one target gene or gene product is induced relative to non-HCC control cells, whereby a diagnosis is, at least in part, afforded.

Preferably, measuring said expression is of two or more target cellular gene sequences. Preferably, measurement of said expression is by use of high-throughput microarray methods.

Polynucleotides and expression vectors. Particular embodiments provide an isolated polynucleotide with a sequence comprising a transcriptional initiation region and a sequence encoding a target gene-specific antisense oligonucleotide at least 10, 15, 17, 20, 22 or 25 nucleotides in length, and a recombinant vector comprising this polynucleotide (e.g., expression vector). Preferably, the transcriptional initiation region is a strong constitutively expressed mammalian pol III-or pol II-specific promoter, or a viral promoter.

### Additional Oligonucleotide Modulators

Included within the scope of the invention are oligonucleotides capable of hybridizing with target gene DNA or RNA, referred to herein as the ‘target’ polynucleotide. An oligonucleotide need not be 100% complementary to the target polynucleotide, as long as specific hybridization is achieved. The degree of hybridization to be achieved is that which interferes with the normal function of the target polynucleotide, be it transcription, translation, pairing with a complementary sequence, or binding with another biological component such as a protein. An antisense oligonucleotide, including a preferred PMO antisense oligonucleotide, can interfere with DNA replication and transcription, and it can interfere with RNA translocation, translation, splicing, and catalytic activity.

The invention includes within its scope any oligonucleotide of about 10 to about 35 nucleotides in length, including variations as described herein, wherein the oligonucleotide hybridizes to a target sequence, including DNA or mRNA, such that an effect on the normal function of the polynucleotide is achieved. The oligonucleotide can be, for example, 10; 15, 17, 20, 22, 23, 25, 30 or 35 nucleotides in length. Oligonucleotides larger than 35 nucleotides are also contemplated within the scope of the present invention, and may for example, correspond in length to a complete target cDNA (i.e., MRNA) sequence, or to a significant or substantial portion thereof.

Antisense oligonucleotides. Examples of representative preferred antisense compounds useful in the invention are based on mRNA sequences encoding a target polypeptide (e.g., SEQ ID NOS:1-7), and include oligonucleotides containing modified backbones or non-natural intemucleoside linkages. Oligonucleotides having modified backbones include those retaining a phosphorus atom in the backbone, and those that do not have a phosphorus atom in the backbone.

Preferred modified oligonucleotide backbones include phosphorothioates or phosphorodithioate, chiral phosphorothioates, phosphotriesters and alkyl phosphotriesters, aminoalkylphosphotriesters, methyl and other alkyl phosphonates including methylphosphonates, 3′-alkylene phosphonates and chiral phosphonates, phosphinates, phosphoroamidates or phosphordiamidates, including 3′-amino phosphoroamidate and aminoalkylphosphoroamidates, and phosphorodiamidate morpholino oligomers (PMOs), thiophosphoroamidates, phosphoramidothioates, thioalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

The antisense oligonucleotide may also comprise at least one modified sugar moiety selected from the group including, but not limited to arabinose, 2-fluoroarabinose, xylulose, hexose and 2′-O-methyl sugar moieties.

The antisense oligonucleotide may comprise at least one modified base moiety which is selected from the group including, but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine (see also U.S. Pat. No. 5,958,773 and patents disclosed therein).

Examples of inventive antisense oligonucleotides of length X (in nucleotides), as indicated by polynucleotide positions with reference to, e.g., SEQ ID NO:9, include those corresponding to sets of consecutively overlapping oligonucleotides of length X, where the oligonucleotides within each consecutively overlapping set (corresponding to a given X value) are defined as the finite set of Z oligonucleotides from nucleotide positions:

n to (n+(X−1));

where n=1, 2, 3, . . . (Y-(X−1));

where Y equals the length (nucleotides or base pairs) of SEQ ID NO:9 (1,890);

where X equals the common length (in nucleotides) of each oligonucleotide in the set (e.g., X=20 for a set of consecutively overlapping 20-mers); and

where the number (Z) of consecutively overlapping oligomers of length X for a given SEQ ID NO of length Y is equal to Y-(X−1). For example Z=1,890−19=1,871 for SEQ ID NO:9, where X=20.

Examples of inventive 20-mer oligonucleotides include the following set of 1,871 oligomers, indicated by polynucleotide positions with reference to SEQ ID NO:9 (PGRMC1 cDNA): 1-20, 2-21, 3-22, 4-23, 5-24, . . . 1,869-1,888, 1,870-1,889 and 1,871-1,890.

Likewise, examples of 25-mer oligonucleotides include the following set of 1,866 oligomers, indicated by polynucleotide positions with reference to SEQ ID NO:9: 1-25, 2-26, 3-27, 4-28, 5-29, . . . 1,864-1,888, 1,865-1,889 and 1,866-1,890.

The present invention encompasses, for each target sequence (e.g., for each nucleotide SEQ ID NOS:9-13 (encoding SEQ ID NOS:1-4 and 5, respectively), multiple consecutively overlapping sets of oligonucleotides or modified oligonucleotides of length X, where, e.g., X=10, 17, 20, 22, 23, 25, 30 or 35 nucleotides.

Various SEQ ID NOS and the associated protein target are listed in Table 1:

TABLE 1
Exemplary protein targets and associated mRNA/cDNA sequences

| | Protein SEQ ID | | | |
|---|---|---|---|---|
| | NO/accession | mRNA SEQ ID | mRNA | Transcript |
| Protein name | number | NO | variants | variants |
| PGRMC1 | SEQ ID NO: 1/ | SEQ ID | BC034238, | |
| (prostaglandin | NP_006658.1 | NO: 9/NM_006667 | CR456993, | |
| receptor | | | Y12711 | |
| membrane | | | | |
| component 1) | | | | |
| SEMA5A | SEQ ID NO: 2/ | SEQ ID | U52840 | BM679516, |
| (semaphorin | NP_003957.1 | NO: 10/NM_003966 | | AL598351, |
| 5A) | | | | AV728993, |
| | | | | CA865957, |
| | | | | AV728562 |
| SLC2A2 | SEQ ID | SEQ ID | J03810, | BG569654, |
| (solute carrier | NO: 3/NP_000331.1 | NO: 11/NM_000340 | BC060041 | AW300621.1, |
| family | | | | BG616475, |
| member) | | | | AV688945.2, |
| | | | | BG564591 |
| ABCC2 (ATP- | SEQ ID | SEQ ID | U49248, | BP276466, |
| binding | NO: 4/NP_000383.1 | NO: 12/NM_000392 | U63970, | CD608372.1, |
| cassette | | | X96395 | CD608373, |
| subfamily C | | | | AV647272.1 |
| member 2) | | | | |
| HAL (histidine | SEQ ID | SEQ ID | D16626 | W69965.1, |
| ammonia | NO: 5/NP_002099.1 | NO: 13/NM_002108 | | AV689503, |
| lyase) | | or ABO42217 | | AV656894.2 |

Representative siRNA sequence regions are disclosed herein, in view of the above algorithm in combination with the teachings on design (e.g., length, structure, composition, etc), preparation and use thereof, provided herein below under “siRNA.”

The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. Thus, the oligonucleotide may include other appended groups such as peptides (e.g., for targeting host cell receptors in vivo), or agents facilitating or modulating transport across the cell membrane (Letsinger et al., Proc. Natl. Acad. Sci. USA 86:6553-6556, 1989; Lemaitre et al., Proc. Natl. Acad. Sci. USA 84:648-652, 1987; PCT WO88/09810, published Dec. 15, 1988) or the blood-brain barrier (PCT WO89/10134, published Apr. 25, 1988), or the nuclear membrane, and may include hybridization-triggered cleavage agents (Krol et al., BioTechniques 6:958-976, 1988) or intercalating agents (Zon, Pharm. Res. 5:539-549, 1988). To this end, the oligonucleotide may be conjugated to another molecule, e.g., a peptide, hybridization-triggered cross-linking agent, transport agent, hybridization-triggered cleavage agent, etc.

Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

Although the inventors are not bound by a particular mechanism of action, it is believed that the antisense oligonucleotides achieve an inhibitory effect by binding to a complementary region of the target polynucleotide within the cell using Watson-Crick base pairing. Where the target polynucleotide is RNA, experimental evidence indicates that the RNA component of the hybrid is cleaved by RNase H (Giles, R. V. et al., Nuc. Acids Res. (1995) 23:954-961; U.S. Pat. No. 6,001,653). Generally, a hybrid containing 10 base pairs is of sufficient length to serve as a substrate for RNase H. However, to achieve specificity of binding, it is preferable to use an antisense molecule of at least 17 nucleotides, as a sequence of this length is likely to be unique among human genes.

Antisense approaches comprise the design of oligonucleotides (either DNA or RNA) that are complementary to the target gene sequence (e.g., mRNA). The antisense oligonucleotides bind to the complementary mRNA transcripts and prevent translation. Absolute complementarily, although preferred, is not required. A sequence “complementary” to a portion or region of the target mRNA, as referred to herein, means a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize depends on both the degree of complementarity and the length of the antisense nucleic acid. Generally, the longer the hybridizing nucleic acid, the more base mismatches with an RNA are accommodated without compromising stable duplex (or triplex, as the case may be) formation. One skilled in the art ascertains a tolerable degree of mismatch by use of standard procedures to determine the melting point of the hybridized complex.

As disclosed in U.S. Pat. No. 5,998,383, incorporated herein by reference, the oligonucleotide is selected such that the sequence exhibits suitable energy related characteristics important for oligonucleotide duplex formation with their complementary targets, and shows a low potential for self-dimerization or self-complementation (Anazodo et al., Biochem. Biophys. Res. Commun. (1996) 229:305-309). The computer program OLIGO (Primer Analysis Software, Version 3.4), is used to determined antisense sequence melting temperature, free energy properties, and to estimate potential self-dimer formation and self-complementarity properties. The program allows the determination of a qualitative estimation of these two parameters (potential self-dimer formation and self-complementary) and provides an indication of “no potential” or “some potential” or “essentially complete potential.” Preferably, segments of target gene sequences are selected that have estimates of no potential in these parameters. However, segments that have “some potential” in one of the categories nonetheless can have utility, and a balance of the parameters is routinely used in the selection.

While antisense nucleotides complementary to the coding region sequence of a mRNA are used in accordance with the invention, those complementary to the transcribed, untranslated region, or translational initiation site region are sometimes preferred. Oligonucleotides that are complementary to the 5′ end of the message, e.g., the 5′-untranslated sequence (up to and including the AUG initiation codon), frequently work most efficiently at inhibiting translation. However, sequences complementary to the 3′-untranslated sequences, or other regions of mRNAs are also effective at inhibiting translation of mRNAs (see e.g., Wagner, Nature 372:333-335, 1994). In the antisense art a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation.

Such experimentation can be performed routinely by transfecting or loading cells with an antisense oligonucleotide, followed by measurement of messenger RNA (mRNA) levels in the treated and control cells by reverse transcription of the mRNA and assaying of respective cDNA levels. Measuring the specificity of antisense activity by assaying and analyzing cDNA levels is an art-recognized method of validating antisense results. Routinely, RNA from treated and control cells is reverse-transcribed and the resulting cDNA populations are analyzed (Branch, A. D., T.I.B.S.(1998) 23:45-50).

According to the present invention, antisense efficacy can be alternately determined by measuring the biological effects on cell growth, phenotype or viability as is known in the art. According to particular aspects of the present invention, cultures of, for example, HCC cells are loaded with inventive oligonucleotides designed to target target gene sequences. The effects of such loading on cell growth, phenotype or viability are measured.

Ribozymes. Modulators of target gene expression may be ribozymes. A ribozyme is an RNA molecule that specifically cleaves RNA substrates, such as mRNA, resulting in specific inhibition or interference with cellular gene expression. As used herein, the term ribozymes includes RNA molecules that contain antisense sequences for specific recognition, and an RNA-cleaving enzymatic activity. The catalytic strand cleaves a specific site in a target RNA at greater than stoichiometric concentration. Preferably the ribozyme is engineered so that the cleavage recognition site is located near the 5′ end of the target mRNA (i.e., to increase efficiency and minimize the intracellular accumulation of non-functional mRNA transcripts).

A wide variety of ribozymes may be utilized within the context of the present invention, including for example, the hammerhead ribozyme (for example, as described by Forster and Symons, Cell (1987) 48:211-220; Haseloff and Gerlach, Nature (1988) 328:596-600; Walbot and Bruening, Nature (1988) 334:196; Haseloff and Gerlach, Nature (1988) 334:585); the hairpin ribozyme (for example, as described by Haseloff et al., U.S. Pat. No. 5,254,678, issued Oct. 19, 1993 and Hempel et al., European Patent Publication No. 0 360 257, published Mar. 26, 1990); and Tetrahymena ribosomal RNA-based ribozymes (see Cech et al., U.S. Pat. No. 4,987,071). The Cech-type ribozymes have an eight-base pair active site that hybridizes to a target RNA sequence whereafter cleavage of the target RNA takes place. Ribozymes of the present invention typically consist of RNA, but may also be composed of DNA, nucleic acid analogs (e.g., phosphorothioates), or chimerics thereof (e.g., DNA/RNA/RNA).

Ribozymes can be targeted to any RNA transcript and can catalytically cleave such transcripts (see, e.g., U.S. Pat. No. 5,272,262; U.S. Pat. No. 5,144,019; and U.S. Pat. Nos. 5,168,053, 5,180,818, 5,116,742 and 5,093,246 to Cech et al.). According to certain embodiments of the invention, any such target gene sequence-specific ribozyme, or a nucleic acid encoding such a ribozyme, may be delivered to a host cell to effect inhibition of target gene expression. Ribozymes and the like may therefore be delivered to the host cells by DNA encoding the ribozyme linked to a eukaryotic promoter (e.g., a strong constitutively expressed pol III- or pol II-specific promoter), or a eukaryotic viral promoter, such that upon introduction into the nucleus, the ribozyme will be directly transcribed.

Triple-helix formation. Alternatively, target gene expression can be reduced by targeting deoxyribonucleotide sequences complementary to the regulatory region of the target gene (e.g., respective promoter and/or enhancers) to form triple helical structures that prevent transcription of the target gene (see, e.g., Helen, Anticancer Drug Des., 6:569-84, 1991; Helene et al., Ann, N.Y. Acad. Sci., 660:27-36, 1992; and Maher, Bioassays 14:807-15, 1992).

siRAA. The invention, in particular aspects, contemplates introduction of RNA with partial or fully double-stranded character into the cell or into the extracellular environment. According to particular aspects of the present invention, inhibition is specific to the particular target cellular gene expression product in that a nucleotide sequence from a portion of the validated sequence is chosen to produce inhibitory RNA. This process is effective in producing inhibition (partial or complete), and is validated gene-specific. In particular embodiments, the target cell containing the validated gene may be a human HCC cell, or a cell subject to HCC.

Methods of preparing and using siRNA are generally disclosed in U.S. Pat. No. 6,506,559, incorporated herein by reference (see also reviews by Milhavet et al., Pharmacological Reviews 55:629-648, 2003; and Gitlin et al., J. Virol. 77:7159-7165, 2003; incorporated herein by reference).

The siRNA may comprise one or more strands of polymerized ribonucleotide, and may include modifications to either the phosphate-sugar backbone or the nucleoside. For example, the phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses of double-stranded material may yield more effective inhibition. Inhibition is sequence-specific in that nucleotide sequences corresponding to the duplex region of the RNA are targeted for genetic inhibition. Nucleic acid containing a nucleotide sequence identical to a portion of the validated gene sequence is preferred for inhibition. RNA sequences with insertions, deletions, and single point mutations relative to the target sequence have also been found to be effective for inhibition. Sequence identity may be optimized by alignment algorithms known in the art and calculating the percent difference between the nucleotide sequences. Alternatively, the duplex region of the RNA may be defined functionally as a nucleotide sequence that is capable of hybridizing with a portion of the target gene transcript.

RNA may be synthesized either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region may be used to transcribe the RNA strand (or strands).

For siRNA (RNAi), the RNA may be directly introduced into the cell (i.e., intracellularly); or introduced extracellularly into a cavity, interstitial space, into the circulation of an organism, introduced orally, or may be introduced by bathing an organism in a solution containing RNA. Methods for oral introduction include direct mixing of RNA with food of the organism, as well as engineered approaches in which a species that is used as food is engineered to express a RNA, then fed to the organism to be affected. Physical methods of introducing nucleic acids include injection directly into the cell or extracellular injection into the organism of an RNA solution.

Inhibition of gene expression refers to the absence (or observable decrease) in the level of protein and/or mRNA product from a target gene target (e.g., inhibition of gene expression may refer to the absence (or observable decrease) in the level of protein (e.g., SEQ ID NOS: 1-7) and/or mRNA product from a target gene). Specificity refers to the ability to inhibit the target gene without manifest effects on other genes of the cell. The consequences of inhibition can be confirmed by examination of the outward properties of the cell or organism or by biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, fluorescence activated cell analysis (FACS), and viral infection, replication, maturation or progression assays as described herein. For RNA-mediated inhibition in a cell line or whole organism, gene expression is conveniently assayed by use of a reporter or drug resistance gene whose protein product is easily assayed. Many such reporter genes are known in the art.

The phosphodiester linkages of natural RNA may be modified to include at least one of a nitrogen or sulfur heteroatom. Modifications in RNA structure may be tailored to allow specific genetic inhibition while avoiding a general panic response in some organisms which is generated by dsRNA. Likewise, bases may be modified to block the activity of adenosine deaminase. RNA may be produced enzymatically or by partial/total organic synthesis, any modified ribonucleotide can be introduced by in vitro enzymatic or organic synthesis.

RNA containing a nucleotide sequence identical to a portion of a particular target gene sequence are preferred for inhibition. RNA sequences with insertions, deletions, and single point mutations relative to the target sequence may be effective for inhibition. Sequence identity may optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of particular validated gene (e.g., src family kinase target gene) sequence is preferred. Alternatively, the duplex region of the RNA may be defined functionally as a nucleotide sequence that is capable of hybridizing with a portion of the particular validated gene transcript (e.g., 400 mM NaCl, 40 mM PIPES pH 6.4, 1 mM EDTA, 50° C. or 70° C. hybridization for 12-16 hours; followed by washing). The length of the identical nucleotide sequences may be at least 20, 25, 50, 100, 200, 300 or 400 bases.

A 100% sequence identity between the RNA and a particular target gene sequence is not required to practice the present invention. Thus the methods have the advantage of being able to tolerate sequence variations that might be expected due to genetic mutation, strain polymorphism, or evolutionary divergence.

Particular target gene sequence siRNA (e.g., those encoding SEQ ID NOS:1-7) may be synthesized by art-recognized methods either in vivo or in vitro. Endogenous RNA polymerase of the cell may mediate transcription in vivo, or cloned RNA polymerase can be used for transcription in vivo or in vitro. For transcription from a transgene in vivo or an expression construct, a regulatory region (e.g., promoter, enhancer, silencer, splice donor and acceptor, polyadenylation) may be used to transcribe the RNA strand (or strands). Inhibition may be targeted by specific transcription in an organ, tissue, or cell type; stimulation of an environmental condition (e.g., infection, stress, temperature, chemical inducers); and/or engineering transcription at a developmental stage or age. The RNA strands may or may not be polyadenylated; the RNA strands may or may not be capable of being translated into a polypeptide by a cell's translational apparatus.

RNA may be chemically or enzymatically synthesized by manual or automated reactions. The RNA may be synthesized by a cellular RNA polymerase or a bacteriophage RNA polymerase (e.g., T3, T7, SP6). The use and production of an expression construct are known in the art (e.g., WO 97/32016; U.S. Pat. Nos. 5,593,874, 5,698,425, 5,712,135, 5,789,214, and 5,804,693; and the references cited therein). If synthesized chemically or by in vitro enzymatic synthesis, the RNA may be purified prior to introduction into the cell. For example, RNA can be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands.

siRNA may be directly introduced into the cell (i.e., intracellularly); or introduced extracellularly into a cavity, interstitial space, into the circulation of an organism, introduced orally, or may be introduced by bathing an organism in a solution containing the RNA. Methods for oral introduction include direct mixing of the RNA with food of the organism, as well as engineered approaches in which a species that is used as food is engineered to express the RNA, then fed to the organism to be affected. For example, the RNA may be sprayed onto a plant or a plant may be genetically engineered to express the RNA in an amount sufficient to kill some or all of a pathogen known to infect the plant. Physical methods of introducing nucleic acids, for example, injection directly into the cell or extracellular injection into the organism, may also be used. Vascular or extravascular circulation, the blood or lymph system, and the cerebrospinal fluid are sites where the RNA may be introduced. A transgenic organism that expresses RNA from a recombinant construct may be produced by introducing the construct into a zygote, an embryonic stem cell, or another multipotent cell derived from the appropriate organism.

Physical methods of introducing nucleic acids include injection of a solution containing the RNA, bombardment by particles covered by the RNA, soaking the cell or organism in a solution of the RNA, or electroporation of cell membranes in the presence of the RNA. A viral construct packaged into a viral particle would accomplish both efficient introduction of an expression construct into the cell and transcription of RNA encoded by the expression construct. Other methods known in the art for introducing nucleic acids to cells may be used, such as lipid-mediated carrier transport, chemical-mediated transport, such as calcium phosphate, and the like. Thus the RNA may be introduced along with components that perform one or more of the following activities: enhance RNA uptake by the cell, promote annealing of the duplex strands, stabilize the annealed strands, or other-wise increase inhibition of the target gene.

The siRNA may be used alone or as a component of a kit having at least one of the reagents necessary to carry out the in, vitro or in vivo introduction of RNA to test samples or subjects. Preferred components are the dsRNA and a vehicle that promotes introduction of the dsRNA. Such a kit may also include instructions to allow a user of the kit to practice the invention.

Suitable injection mixes are constructed so animals receive an average of 0.5×10⁶ to 1.0×10⁶ molecules of RNA. For comparisons of sense, antisense, and dsRNA activities, injections are compared with equal masses of RNA (i.e., dsRNA at half the molar concentration of the single strands). Numbers of molecules injected per adult are given as rough approximations based on concentration of RNA in the injected material (estimated from ethidium bromide staining) and injection volume (estimated from visible displacement at the site of injection). A variability of several-fold in injection volume between individual animals is possible.

### Particular Specific Embodiments

Particular aspects provide a method for treating or preventing hepatocellular carcinoma, comprising administering to a subject in need thereof a therapeutic agent in an amount sufficient to inhibit the expression or biological activity of at least one polypeptide selected from the group consisting of SEQ ID NOS:1-7, and naturally occurring variants thereof. Preferably, the therapeutic agent comprises comprises at least one agent selected from the group consisting of: a polyclonal antibody; a monoclonal antibody; a single chain Fv, a Fab fragment, a Fab(2) fragment, a minibody or a domain-deleted antibody; a cytokine, chemokine, growth factor or other naturally occurring ligand; and a synthetic molecule.

Additional embodiments provide a method for treating or preventing hepatocellular carcinoma, comprising generating in a subject in need thereof an immune response directed against at least one polypeptide selected from the group consisting of: SEQ ID NOS:1-7, wherein the method comprises immunizing the patient with one or more of the polypeptides or immunogenic fragments thereof in an amount sufficient to illicit an immune response. Preferably, the method comprises inhibition of the biological activity of the polypeptide of SEQ ID NO:1, SEQ ID NO:2, or of both, and wherein the therapeutic agent comprises at least one agent selected from the group consisting of: a polypeptide that is at least 88% identical at the amino acid level to that of SEQ ID NO:1 or SEQ ID NO:2; a polypeptide fragment comprising at least 15 contiguous amino acids of SEQ ID NO:1 or SEQ ID NO:2; a naturally occurring allelic variant of SEQ ID NO:1 or SEQ ID NO:2 that is encoded by a nucleic acid molecule that is at least 88% identical at the oligonucleotide level to a gene encoding SEQ ID NO:1 or SEQ ID NO:2; a polypeptide fragment of a naturally occurring allelic variant of SEQ ID NO:1 or SEQ ID NO:2, wherein the fragment comprises at least 15 contiguous amino acids of SEQ ID NO:1 or SEQ ID NO:2; and a chimeric polypeptide comprising polypeptide fragments of SEQ ID NO:1 or SEQ ID NO:2, wherein the polypeptide fragments are linked in a manner sufficient to mimic a ligand binding site of SEQ ID NO:1 or SEQ ID NO:2, and wherein the therapeutic agent exhibits the ligand binding activity of SEQ ID NO:1 or SEQ ID NO:2.

Yet additional aspects provide a method for treating or preventing hepatocellular carcinoma, comprising administering to a subject in need thereof, a therapeutic compound comprising a targeting agent conjugated or coupled to a therapeutic moiety, wherein the targeting agent binds a polypeptide selected from the group consisting of SEQ ID NOS:1-7, and wherein the therapeutic moiety is cytotoxic or cytostatic. Preferably, the targeting agent comprises at least one therapeutic moiety selected from the group consisting of: a polyclonal antibody; a monoclonal antibody; a single chain Fv, a Fab fragment, a Fab(2) fragment, a minibody or a domain-deleted antibody; a bifunctional chimeric antibody molecule; a cytokine, chemokine, growth factor or other naturally occurring ligand; and a synthetic molecule. Preferably, the therapeutic moiety comprises at least one of: an antibiotic; a toxin; an apoptotic agent; an antimetabolite; a growth factor or cytokine; an RNase; and an anti-angiogenic agent.

Further embodiments provide a method for treating or preventing hepatocellular carcinoma, comprising administering to a subject in need thereof, a therapeutic agent that reduces the physiological levels of at least one polypeptide selected from the group consisting of SEQ ID NOS: 1-7. Preferably, the therapeutic agent is an antisense polynucleotide administered to inhibit expression of a gene, or translation of a respective mRNA encoding the at least one polypeptide. Preferably, the antisense molecule is a polynucleotide comprising at least 10 contiguous nucleotides complementary to a sequence that encodes the at least one polypeptide. Preferably, the antisense molecule is a peptide polynucleic acid or a non-nucleic acid polymer, and wherein the antisense molecule is complementary to at least 10 contiguous nucleotides of the at least one polypeptide. Preferably, the non-nucleic acid polymers are selected from the group consisting of phosphorothionate derivatives, morpholino oligonucleotides, and combinations thereof. In particular aspects, the therapeutic agent is a ribozyme.

Yet further embodiments provide a method for treating or preventing hepatocellular carcinoma, comprising administering to a subject in need thereof a therapeutic agent to increase histidine ammonia lyase activity in the subject. Preferably, the therapeutic agent is a polynucleotide that encodes a polypeptide or polypeptide fragment comprising at least 15 contiguous amino acids that has at least 88% sequence identity to the polypeptide of SEQ ID NO:8. Preferably, the therapeutic agent is a polypeptide or polypeptide fragment comprising at least 15 contiguous amino acids that has at least 88% sequence identity to the polypeptide of SEQ ID NO:8.

Additional aspects provide a method of treating or preventing hepatocellular carcinoma (HCC), comprising administering to a subject in need thereof a therapeutic agent that is an anti-histamine.

In particular aspects, the above-described methods additionally comprise at least one step selected from the group consisting of: administering a chemotherapeutic agent; administering radiation therapy; administering surgical resection or liver transplantation; administering radio frequency ablation; administering cryosurgery; administering ethanol ablation; and administering embolization.

In yet additional aspects, the above-described methods are conducted prophylactically.

Further embodiments provide a method for identification of a therapeutic agent for the treatment or prevention of hepatocellular carcinoma, comprising: contacting at least one polypeptide selected from the group consisting of SEQ ID NOS: 1-5 with a test compound; and determining, using one or more suitable assays, the effect of the test compound on the activity of the at least one polypeptide by comparison with a control to identify a test compound that modulates the activity of the at least one polypeptide. Preferably, determining in b) comprises detecting binding of the test compound to the at least one polypeptide, and wherein the binding is detected by at least one method selected from the group consisting of: direct detection of test compound binding to the at least one polypeptide; competition binding assay; and an assay for an activity mediated by the at least one polypeptide.

Yet further aspects provide a pharmaceutical composition, comprising, in combination with a pharmaceutically acceptable carrier or excipient, at least one agent suitable for treating or preventing hepatocellular carcinoma (HCC), wherein the agent is selected from the group consisting of: an antibody or antibody reagent specific for at least one polypeptide selected from the groups consisting of SEQ ID NOS:1-7; an antisense molecule specific for at least one sequence selected from the group consisting of SEQ ID NOS:9-13; an siRNA agent specific for at least one sequence selected from the group consisting of SEQ ID NOS:9-12; a soluble receptor corresponding to at least one polypeptide selected from the groups consisting of SEQ ID NOS:1-7; and a polynucleotide encoding HAL.

Additional aspect provide for use of the pharmaceutical composition of claim 22 in preparing a medicament for treating or preventing hepatocellular carcinoma (HCC).

No license is expressly or implicitly granted to any patent or patent applications referred to or incorporated herein. The discussion above is descriptive, illustrative and exemplary and is not to be taken as limiting the scope of any aspect of the inventive subject matter defined by any presently or subsequently appended claims.

### Particular References Cited:

1) Adams, J. C. and Tucker, R. P. (2000) Dev. Dyn. 218: 280-299

2) Agrawal, S. and lyer, R. P. (1997) Pharmacol. Ther. 76:151-160

3) Ajani, J. A. et al. (2000) Cancer J. 6: 78-81

4) Aoki, Y. and Tanaka, K. -(2002) Expert Rev. Anticancer Ther. 1: 73-82

5) Baselga, J. et al. (1998) Can. Res. 13: 2325-2831

6) Befeler, A. S. and Bisceglie, A. M. (2002) Gastroenterol. 122: 1609-1619

7) Behr, T. et al. (1995) Can. Res. 55: 5777s-5785s

8) Bernstein, I. D. (2000) Leukemia 14:474-475

9) Brinkmann, U. et al. (2001) Expert. Opin. Biol. Ther. 1: 693-702

10) Bruix, J. et al. (2001) J. Hepatol. 35: 421-430

11) Busto, G. et al. (2001) Expert Rev. Anticancer Ther. 1: 111-115

12) Carter, P. et al. (1992) Proc. Natl. Acad. Sci. USA 89: 4285-4289

13) Chapman, A. P. (2002) Adv. Drug Deliv. Rev. 54: 531-545

14) Chester, K. A. et al. (2000) Cancer Chemother. Pharmacol. 46 (suppl.): S8-S12

15) Clynes, R. A. et al. (2000) Nat. Med. 6: 443-446

16) Cohen, J. S. (1994) Adv. Pharmacol. 25: 319-339

17) Crombet-Ramos, T. et al. (2002) Int. J. Cancer 101: 567-575

18) Czuczman, M. S. et al. (1999) J. Clin. Oncol. 17: 268-276

19) Dani, M. (2001) J. Recept. Signal Transduct. Res. 21: 469-488

20) Davis, C. G. et al. (1999) Cancer Metastisis Rev. 18: 421-425

21) Deckert, P. M. et al. (2000) Int. J. Cancer 87: 382-390

22) Denny, W. A. (2001) Eur. J. Med. Chem. 36: 577-595

23) Eberwine, J. (1996) Biotechniques 20: 584-591

24) Evers, R. et al. (1998) J. Clin. Invest. 101: 1310-1319

25) Felip, E. and Rosell, R. (2001) Expert Rev. Anticancer Ther. 1: 224-228

26) Floyd, C. D. et al. (1999) Prog. Med. Chem. 36: 91-168

27) Frankel, A. E. et al. (2000) Clin. Can. Res. 6: 326-334

28) Garnett, M. C. (2001) Adv. Drug Deliv. Res. 53:171-216

29) Geiss, G. K. et al. (2000) Virol. 266: 8-16

30) Geiss, G. K. et al. (2001) J. Virol. 75: 4321-4331

31) Gentry, L. E. and Lawton, A. (1986) Virology 152: 421-431

32) Gerk, P. M. and Vore, M. (2002) J. Pharmacol. Exp. Ther. 302: 407-415

33) Glennie, M. J. and Johnson, W. M. (2000) Immunol. Today 21: 403-410

34) Goldenberg, M. M. (1999) Clin. Ther. 21: 309-318

35) Greaves, M. W. (2001) Dermatol. Clin. 19: 53-62

36) Green, L. L. (1999) J. Immunol. Methods 231: 11-23

37) Green, M. C. (2000) Cancer Treat. Rev. 26: 269-286

38) Guha, C. et al. (2001) J. Hepatobiliary Pancreat. Surg. 8: 51-57

39) Harvey, A. L. (1999) TIPS 20: 196-198

40) Hortobagyi, G. N. (2002) 29 (suppl. 11): 134-144

41) Hu, S. et al. (1996) Can. Res. 56: 3055-3061

42) Huston, J. S. and George, A. J. (2001) Hum. Antibodies 10: 127-142

43) lyer, R. P. et al. (1990) Nucleic Acids Res. 18: 2855-2859

44) Jones, P. T. et al. (1986) Nature 321: 522-525

45) Johnson, P. W. and Glennie, M. J. (2001) 85:1619-1623

46) Khare, P. D. (2001) Cancer Res. 61: 370-375

47) Kim, R. et al. (2002) Anticancer Res. 22: 2413-2418

48) King, D. J. et al. (1994) Cancer Res. 54: 6176-6185

49) Kluth, D. C. and Rees, A. J. (1996) Semin. Nephrol. 16: 576-582

50) Kortt, A. A. (2001) Biomol. Eng. 18: 95-108

51) Krebs, C. J. (2000) Proc. Natl. Acad. Sci U.S.A. 97:12816-12821

52) Kreitman, R. J. (2001 a) Curr. Pharm. Biotechnol. 2: 313-325

53) Kreitman, R. J. (2001 b) Curr. Opin. Investig. Drugs 2: 1282-1293

54) Kriangkum, J. et al. (2001) Biomol. Eng. 18: 31-40

55) Landro, J. A. (2000) J. Pharmacol. Toxicol. Methods 44: 273-289

56) Leong, S. R. et al. (2001) Cytokine 16: 106-119

57) Lennon, G. et al. (1996) Genomics 33(1): 151-152

58) Lutzky, J. et al. (2002) Semin. Oncol. 29: 462-470

59) Lowry, S. F. (1993) New Horiz. 1:120-126

60) Maloney, D. G. (2001) Anticancer Drugs 12 (suppl. 2): S1-4

61) Maloney et al. (2002) Semin. Oncol. 29 (suppl. 2): 2-9

62) Marty, C. et al. (2002) Br. J. Cancer 87: 106-112

63) Mazzolini, R. J. et al. (2001) Dig. Dis. 19: 324-332

64) McGinn, C. J. et al. (2002) Cancer 95: 933-940

65) McClay, E. F. (2002) Semin. Oncol. 29: 389-399

66) Merdan, T. et al. (2002) Adv. Drug Deliv. Rev. 54: 715

67) Morrison, S. L. et al. (1984) Proc. Natl. Acad. Sci. U.S.A. 81:6851 68555

68) Munro, M. H. et al. (1999) J. Biotechnol. 70:15-25

69) Murray, K. M and Dahl S. L. (1997) Annal. Pharmacother. 31:1335-1338

70) Nakamura, T. et al. (2000) Int. J. Immunopharmacol. 22: 131-141

71) Newman, R. et al. (1992) Biotechnol. 10: 1455-1460

72) Newton, D. L. and Ryback, S. M. (2001) Expert. Opin. Biol. Ther. 1: 995-1003

73) Nuttall, S. D. et al. (2000) Curr. Pharm. Biotechnol. 1: 253-263

74) Oka, Y. et al. (1990) Nature 345: 550-553

75) Olive, D. et al. (1986) Eur. J. Immunol. 16: 611-616

76) Park, J. W. and Smolen, J. (2001) Adv. Prot. Chem. 56: 369-421

77) Park, J. W. et al. (2002) Clin. Cancer Res. 8: 1172-1181

78) Perales, M. A. and Wolchok, J. D. (2002) Cancer Invest. 20:1012-1026

79) Permutt, M. A. et al. (1989) Proc. Natl. Acad. (USA) 86:8688-8692

80) Ramstrom, O. and Lehn, J. M. (2002) Nat. Rev. Drug Discov. 1: 26-36

81) Reff, M. E. and Heard, C. (2001) Crit. Rev. Oncol. Hematol. 40: 25-35

82) Reiter, Y. (2001) Adv. Can. Res. 81: 93-124

83) Rhyner, C. et al. (2002) Curr. Pharm. Biotechnol. 3: 13-21

84) Schmitz, V. et al. (2002) Gut 1: 30-35

85) Siegel, D. L. (2002) Transfus. Clin. Biol. 9: 15-22

86) Shusta, E. V. et al. (1999) Curr. Opin. Biotechnol. 10: 117-122

87) Suchi, M. et al. (1995) Genomics 29: 98-104

88) Suh, B. C. et al. (2001) J. Immunol. 167:1663-1671

89) Tramontano, A. et al. (1994) J. Mol. Recognit. 1: 9-24

90) Vidovic, D. et al. (2002) Int. J. Cancer 102: 660-664

91) Walsh, G. M. et al. (2001) Drugs 61: 207-236

92) Weir, A. N. C. et al. A. N. C. (2002) Bichem Soc. Trans. 30: 512-516

93) Witzig, T. E. (2001) Cancer Chemother. Pharmacol. 48 (suppl.1): S91-S95

94) Wu, J. et al (2002) Front. Biosci. 7: d717-d725

95) Wuest, T. et al. (2002) Oncogene 21: 4257-4265

96) Xiang, J. et al. (1997) J. Biotechnol. 53: 3-12

97) Xu, G. and McLeod, H. L. (2001) Clin. Cancer Res. 7: 3314-3324

100) Yang, X. D. et al. (1999) J. Leukoc. Biol. 66: 401-410

101) Yang, X. D. et al. (2001) Crit. Rev. Oncol. Hematol. 38: 17-23

102) Yoon, D. Y. and Dinarello, C. A. (1998) J. Immunol. 160: 3170-3179

1. A method for treating or preventing hepatocellular carcinoma, comprising administering to a subject in need thereof a therapeutic agent in an amount sufficient to inhibit the expression or biological activity of at least one polypeptide selected from the group consisting of SEQ ID NOS:1-7, and naturally occurring variants thereof.

2. The method of claim 1, wherein the therapeutic agent comprises at least one agent selected from the group consisting of: a polyclonal antibody; a monoclonal antibody; a single chain Fv, a Fab fragment, a Fab(2) fragment, a minibody or a domain-deleted antibody; a cytokine, chemokine, growth factor or other naturally occurring ligand; and a synthetic molecule.

3. A method of treating or preventing hepatocellular carcinoma, comprising generating in a subject in need thereof an immune response directed against at least one polypeptide selected from the group consisting of: SEQ ID NOS:1-7, wherein the method comprises immunizing the patient with one or more of the polypeptides or immunogenic fragments thereof in an amount sufficient to illicit an immune response.

4. The method of claim 1, comprising inhibition of the biological activity of the polypeptide of SEQ ID NO:1, SEQ ID NO:2, or of both, and wherein the therapeutic agent comprises at least one agent selected from the group consisting of: a polypeptide that is at least 88% identical at the amino acid level to that of SEQ ID NO:1 or SEQ ID NO:2; a polypeptide fragment comprising at least 15 contiguous amino acids of SEQ ID NO:1 or SEQ ID NO:2; a naturally occurring allelic variant of SEQ ID NO:1 or SEQ ID NO:2 that is encoded by a nucleic acid molecule that is at least 88% identical at the oligonucleotide level to a gene encoding SEQ ID NO:1 or SEQ ID NO:2; a polypeptide fragment of a naturally occurring allelic variant of SEQ ID NO:1 or SEQ ID NO:2, wherein the fragment comprises at least 15 contiguous amino acids of SEQ ID NO:1 or SEQ ID NO:2; and a chimeric polypeptide comprising polypeptide fragments of SEQ ID NO:1 or SEQ ID NO:2, wherein the polypeptide fragments are linked in a manner sufficient to mimic a ligand binding site of SEQ ID NO:1 or SEQ ID NO:2, and wherein the therapeutic agent exhibits the ligand binding activity of SEQ ID NO:1 or SEQ ID NO:2.

5. A method of treating or preventing hepatocellular carcinoma, comprising administering to a subject in need thereof, a therapeutic compound comprising a targeting agent conjugated or coupled to a therapeutic moiety, wherein the targeting agent binds a polypeptide selected from the group consisting of SEQ ID NOS: 1-7, and wherein the therapeutic moiety is cytotoxic or cytostatic.

6. The method of claim 5, wherein the targeting agent comprises at least one therapeutic moiety selected from the group consisting of: a polyclonal antibody; a monoclonal antibody; a single chain Fv, a Fab fragment, a Fab(2) fragment, a minibody or a domain-deleted antibody; a bifunctional chimeric antibody molecule; a cytokine, chemokine, growth factor or other naturally occurring ligand; and a synthetic molecule.

7. The method of claim 5, wherein the therapeutic moiety comprises at least one of: an antibiotic; a toxin; an apoptotic agent; an antimetabolite; a growth factor or cytokine; an RNase; and an anti-angiogenic agent.

8. A method of treating or preventing hepatocellular carcinoma, comprising administering to a subject in need thereof, a therapeutic agent that reduces the physiological levels of at least one polypeptide selected from the group consisting of SEQ ID NOS:1-7.

9. The method of claim 8, wherein the therapeutic agent is an antisense polynucleotide administered to inhibit expression of a gene, or translation of a respective mRNA encoding the at least one polypeptide.

10. The method of claim 9, wherein the antisense molecule is a polynucleotide comprising at least 10 contiguous nucleotides complementary to a sequence that encodes the at least one polypeptide.

11. The method of claim 9; wherein the antisense molecule is a peptide polynucleic acid or a non-nucleic acid polymer, and wherein the antisense molecule is complementary to at least 10 contiguous nucleotides of the at least one polypeptide.

12. The method of claim 11, wherein the non-nucleic acid polymers are selected from the group consisting of phosphorothionate derivatives, morpholino oligonucleotides, and combinations thereof.

13. The method of claim 8, wherein the therapeutic agent is a ribozyme.

14. A method of treating or preventing hepatocellular carcinoma, comprising administering to a subject in need thereof a therapeutic agent to increase histidine ammonia lyase activity in the subject.

15. The method of claim 14, wherein the therapeutic agent is a polynucleotide that encodes a polypeptide or polypeptide fragment comprising at least 15 contiguous amino acids that has at least 88% sequence identity to the polypeptide of SEQ ID NO:8.

16. The method of claim 14, wherein the therapeutic agent is a polypeptide or polypeptide fragment comprising at least 15 contiguous amino acids that has at least 88% sequence identity to the polypeptide of SEQ ID NO:8.

17. A method of treating or preventing hepatocellular carcinoma (HCC), comprising administering to a subject in need thereof a therapeutic agent that is an anti-histamine.

18. The method of any one of claims 1, 3, 5, 8, 14 and 17, further comprising at least one step selected from the group consisting of: administering a chemotherapeutic agent; administering radiation therapy; administering surgical resection or liver transplantation; administering radio frequency ablation; administering cryosurgery; administering ethanol ablation; and administering embolization.

19. The method of any one of claims 1, 3, 5, 8, 14 and 17, wherein the method is conducted prophylactically.

20. A method for identification of a therapeutic agent for the treatment or prevention of hepatocellular carcinoma, comprising:
a) contacting at least one polypeptide selected from the group consisting of SEQ ID NOS:1-5 with a test compound; and
b) determining, using one or more suitable assays, the effect of the test compound on the activity of the at least one polypeptide by comparison with a control to identify a test compound that modulates the activity of the at least one polypeptide.

21. The method of claim 20, wherein determining in b) comprises detecting binding of the test compound to the at least one polypeptide, and wherein the binding is detected by at least one method selected from the group consisting of: direct detection of test compound binding to the at least one polypeptide; competition binding assay; and an assay for an activity mediated by the at least one polypeptide.

22. A pharmaceutical composition, comprising, in combination with a pharmaceutically acceptable carrier or excipient, at least one agent suitable for treating or preventing hepatocellular carcinoma (HCC), wherein the agent is selected from the group consisting of: an antibody or antibody reagent specific for at least one polypeptide selected from the groups consisting of SEQ ID NOS:1-7; an antisense molecule specific for at least one sequence selected from the group consisting of SEQ ID NOS:9-13; an siRNA agent specific for at least one sequence selected from the group consisting of SEQ ID NOS:9-12; a soluble receptor corresponding to at least one polypeptide selected from the groups consisting of SEQ ID NOS: 1-7; and a polynucleotide encoding HAL.

23. (canceled)
